(19) United States
(12) Patent Application Publication
(10) Pub. No.: US 2001/0044413 A1
(43) Pub. Date: Nov. 22, 2001

(54) In situ bioreactors and methods of use thereof

(76) Inventors: Glenn Pierce, Rancho Santa Fe, CA (US); Lois Ann Chandler, Encinitas, CA (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 09/729,644

(22) Filed: Nov. 30, 2000

Related U.S. Application Data
(60) Provisional application No. 60/168,470, filed on Dec. 1, 1999.

(51) Int. Cl. A61K 48/00

(52) U.S. Cl. 514/44

(57) ABSTRACT

The present invention provides in situ bioreactors comprising a biocompatible substance comprising nucleic acid molecules and capable of cellular ingrowth and systemic delivery of a bioactive agent. Also provided are compositions, devices, and kits comprising the same. In various embodiments the biocompatible substance comprises a matrix and at least one nucleic acid molecule encoding a bioactive agent. In other embodiments bioreactors are provided wherein a first gene that encodes a growth factor is present and a second gene encoding a bioactive agent is present during manufacture or provided to the bioreactor following manufacture or implantation.

### CROSS-REFERENCE TO RELATED APPLICATION

This application claims priority to U.S. Provisional Application No. 60/168,470, filed Dec. 1, 1999.

### TECHNICAL FIELD

The present invention relates generally to methods, compositions and devices for systemic delivery of a bioactive agent, and in particular to methods, compositions, and devices that utilize nucleic acid delivery through a biocompatible substance (e.g., devices and matrices) capable of supporting cellular ingrowth.

### BACKGROUND OF THE INVENTION

Protein deficiencies lead to a variety of disease states, including, for example, hemophilia, cystic fibrosis, diabetes, anemia, immune disorders (e.g., adenosine deaminase deficiency), and hypercholesterolemia. Accordingly, many clinical conditions, deficiencies, and disease states can be remedied or alleviated by supplying to the patient bioactive agents produced by living cells or removing from the patient deleterious factors which affect living cells. In many cases, these agents can restore or compensate for the impairment or loss of organ or tissue function. The impairment or loss of organ or tissue function may result in the loss of additional metabolic functions. For example, in fulminant hepatic failure, liver tissue is rendered incapable of removing toxins, excreting the products of cell metabolism, and secreting essential products, such as albumin and Factor VIII (Bontempo et al., Blood 69:1721-1724, 1987). Gene therapy offers the promise of alleviating such diseases as well as other conditions such as cancer and viral infections, by providing somatic cells with a nucleic acid molecule encoding a bioactive agent targeted to the condition to be treated. Essentially, gene transfer involves delivery, to target cells, of an expression cassette made up of one or more nucleic acid molecules comprising regulatory elements and therapeutic nucleic acids. Therapeutic nucleic acids may be active as polynucleotides (i.e., antisense or ribozymes), or they may encode a therapeutic polypeptide. Gene transfer can be carried out ex vivo by a procedure in which the cassette is transferred to cells in the laboratory and the genetically modified cells administered to the patient. Alternatively, gene transfer can proceed in vivo. However, the shortcomings of current gene therapeutic methods are well documented and include low transfection efficiency, low expression level of the transferred gene, and lack of prolonged gene expression.

In certain cases, these bioactive agents are biological response modifiers, such as lymphokines or cytokines, which enhance the patient's immune system or act as anti-inflammatory agents. These can be particularly useful in individuals with a chronic parasitic or infectious disease, and may also be useful for the treatment of certain cancers. It may also be desirable to supply trophic factors to a patient, such as nerve growth factor or insulin-like growth factor-one or -two (IGF1 or IGF2). In other embodiments, the biologically active moiety can be a secretory substance, such as a neurotransmitter, neuromodulator, hormone, trophic factor, or growth factor, or a neuroactive substance for the reduction of pain sensitivity. Such neuroactive substances include catecholamines, enkephalins, and opioid peptides.

Many other diseases are, likewise, characterized by a deficiency in a biologically active moiety that cannot easily be supplemented by injections or longer-term, controlled drug release therapies, but instead respond more readily to biological (e.g., receptor signal induced) up- or downregulation as needed. Still other diseases, while not characterized by substance deficiencies, can be treated with bioactive agents normally made and secreted by cells. Thus, trophic and growth factors may be used to prevent neurodegenerative conditions, such as Huntington's and Alzheimer's diseases, and adrenal chromaffin cells which secrete catecholamines and enkephalins, may be used to treat pain.

Infectious diseases, including several types of hepatitis and herpesvirus infections are suitable targets for gene therapy approaches. Such treatment can be effectuated by providing mutant proteins that inhibit viral replication, antisense RNA that blocks translation of viral gene products, ribozymes that attack viral RNA in a specific manner, decoy RNA molecules that efficiently compete for binding of viral proteins, and expression of single chain antibodies that bind to key viral enzymes and prevent functioning.

Artificial organs offer the possibility of sustained systemic delivery of bioactive agents. However, to date, no artificial organ (organoid) has proven clinically successful. The use of neovascularized organoids for gene therapy applications has been proposed by Thompson et al., Proc. Natl. Acad. Sci. USA 86:7928-7932, 1989 and Thompson et al., Science 241:1349-1352, 1988, using ex vivo modified cells. To date, all such organoids have been engineered ex vivo with genetically modified cells. For example, poly(tetrafluoroethylene) fibers have been used as scaffolds onto which a solution containing ex vivo modified cells in a collagen matrix is plated (Dwarki et al., Proc. Natl. Acad. Sci. USA 92:1023-1027, 1995; Naffakh et al., Proc. Natl. Acad. Sci. USA 92:3194-3198, 1995). Use of such ex vivo manipulated cells is logistically difficult, time consuming, and likely cost prohibitive for large scale therapeutics. Such manipulations would necessarily need to be performed by a highly trained technical staff on a patient by patient basis.

Accordingly, there is a need in the art for sustained and controlled gene delivery as well as sustained product expression utilizing in vivo transfer and expression of desired nucleic acid molecules. The current invention meets this need and provides other related advantages.

### SUMMARY OF THE INVENTION

The present invention generally provides an in situ bioreactor adapted for the systemic delivery of a bioactive agent as well as methods and kits related to the same. In one aspect, the invention provides an in situ bioreactor adapted for systemic delivery of bioactive agents, comprising a first nucleic acid molecule encoding a cell growth stimulating agent, a second nucleic acid molecule encoding a bioactive agent, and a biocompatible substance, said substance capable of cellular infiltration. In one embodiment, the cell growth stimulating agent may be a transcription factor, a chemotactic factor, an angiogenic factor, an antisense molecule, a ribozyme, an anti-apoptotic molecule, a growth factor, a cytokine, an extracellular matrix moleule, a cell adhesion protein, a cell retention agent, or a cell surface receptor. In certain embodiments the growth factor may be a member of a number of growth factor families including the transforming growth factor (TGF) family, the fibroblast growth factor (FGF) family, the platelet derived growth factor (PDGF) family, the insulin like growth factor (IGF) family, the vascular endothelial growth factor (VEGF) family, the hepatocyte growth factor (HGF) family, the epidermal growth factor (EGF) family, the colony stimulating factor (CSF) family, the angiopoietin family, the interleukin family or the bone morphogenic factor (BMP) family.

In certain embodiments the cell growth stimulating agent is a ribozyme, antisense or anti-apoptotic agent. In the various embodiments the anti-apoptotic agent is a Bcl-2 family member or homolog thereof. In other embodiments the cell growth stimulating agent is a transcription factor such as NF-κB, E2F, DP1, AP-1, AP-2, myc, p53, Sp1, NFAT, CBP, C/EBP, and nuclear hormone receptor family members.

In yet other embodiments, the bioreactor further comprises a cell retention agent or nucleic acid encoding the same. In the various embodiments the cell retention agent may be magrophage migration inhibitory factor (MIF), extracellular matrix molecules, and cell adhesion molecules.

In further embodiments the second nucleic acid molecule encodes a hormone or other bioactive agent such as growth hormone, inhibin, relaxin, activin, insulin, atrial natriuretic peptide (ANP), luteinizing hormone, follicle-stimulating hormone, releasing hormones, and follitropin, Factor V (FV), Factor VII (FVII), Factor VIII (FVIII), Factor IX (FIX), Factor X, (FX), Factor XI (FXI), Factor XIII (FXIII), erythropoietin (EPO), growth hormone (GH), adenosine deaminase, thrombopoietin, purine nucleoside phosphorylase (PNP), Protein C, Protein S, fibrinolytic agents, an interleukin, an interferon, a globin, an antibody, or an antibody fragment.

In additional embodiments, the first and second nucleic acid molecules are operably linked to promoters. In specific embodiments, the promoters may be independently selected from group consisting of constitutive, inducible, event-specific, and tissue specific promoters. Further, in various embodiments the nucleic acid molecule is in the form of a plasmid, or a recombinant insert in the genome of a virus or associated with a condensing agent.

In another embodiment, at least one nucleic acid molecule is associated with a cell surface binding moiety, such as a ligand for a cell surface receptor. Such ligands may include among other things, polypeptides reactive with the fibroblast growth factor receptor family.

In the various embodiments the biocompatible substance is either a natural (biological) or synthetic matrix. In related embodiments the biological matrix may be collagen, purified proteins, purified peptides, polysaccharides, glycosaminoglycans, and extracellular matrix compositions. In further embodiments the matrix may be fibrin, chitosan, alginate, dextran, hyaluronic acid, cellulose, polyesters, polyethers, polyanhydrides, polyalkylcyanoacrylates, polyacrylamides, polyorthoesters, polyphosphazenes, polyvinylacetates, block copolymers, polypropylene, polytetrafluoroethylene (PTFE), and polyurethanes.

In yet additional embodiments the matrix may be biodegradable or non-biodegradable and may comprise collagen, metal, hydroxyapatite, bioglass, aluminate, bioceramic materials, hyaluronic acid polymers, alginate, acrylic ester polymer, lactic acid polymer, glycolic acid polymer, lactic acid/glycolic acid polymer, purified proteins, purified peptides, and extracellular matrix compositions.

In other embodiments the present invention provides a bioreactor having a biocompatible substance associated with an implantable device. Such devices may be include among other things, a stent, a catheter, a fiber, a hollow fiber, a patch, and a suture.

In another aspect the present invention provides a method adapted for systemic delivery of a protein from a tissue site in an animal, comprising contacting the tissue site with any of the described in situ bioreactors. In the various embodiments the tissue site is a site of an iatrogenic injury, traumatic injury, disease induced injury, or the like. In other embodiments the tissue site is subcutaneous, intramuscular, intraperitoneal, or retroperitoneal and may be in an organ.

In certain embodiments subsequent to contacting the tissue site with the bioreactor, the bioreactor is supplemented with additional quantities of the first nucleic acid molecule encoding a cell growth stimulating agent and/or the second nucleic acid molecule encoding a bioactive agent. In related embodiments, prior to contacting the tissue site with the bioreactor, the bioreactor comprises cells transduced with the first nucleic acid, the second nucleic acid, or both the first and second nucleic acids.

In further aspects a method adapted for systemic delivery of a protein from a tissue site, comprising introducing into a tissue site of an animal an in situ bioreactor, the bioreactor comprising a first nucleic acid molecule encoding a cell growth stimulating agent, wherein the bioreactor comprises a biocompatible substance capable of infiltration by cells, and wherein a second nucleic acid molecule encoding a serum soluble protein is introduced into the bioreactor following cellular infiltration, is provided.

In yet an additional aspect a method adapted for systemic delivery of a protein from a tissue site, comprising contacting a tissue site of an animal with an in situ bioreactor, wherein the bioreactor comprises a biocompatible substance and a first nucleic acid molecule, wherein the first nucleic acid molecule encodes a cell growth stimulating agent, and wherein said encoded cell growth stimulating agent conditions matrix infiltrating cells for uptake of a second nucleic acid molecule encoding a serum soluble protein, is provided. In the various embodiments, the cells are selected from stem cells, macrophages, fibroblasts, or vascular cells and the nucleic acid molecule may be absorbed in, adsorbed to, or impregnated within a biocompatible substance.

Another aspect of the present invention is a method adapted for systemic delivery of a protein from a tissue site, comprising introducing into a tissue site of an animal an in situ bioreactor, wherein the bioreactor comprises a biocompatible substance, a cell growth stimulating agent, and a first nucleic acid molecule encoding a serum soluble protein wherein the bioreactor is capable of infiltration by cells.

In other aspects, the present invention provides, a bi-gene device comprising a biocompatible substance capable of cellular infiltration, a first nucleic acid molecule encoding a growth stimulating agent, and a second nucleic acid molecule encoding a bioactive agent. In certain embodiments, the biocompatible substance associated with the device may comprise a substance selected from PTFE, expanded PTFE, Dacron, metal, polylactic acid, polyglycolic acid, polylactic-polyglycolic acid (PLGA), collagen, bioceramic materials, alginate, or hyaluronic acid.

Also provided are kits for the production of a device comprising an appropriate container, a biocompatible substance, a first nucleic acid molecule encoding a growth stimulating agent and a second nucleic acid molecule encoding a bioactive agent or coated device comprising a device coated with a biocompatible substance, a first nucleic acid molecule encoding a growth stimulating agent, and a second nucleic acid molecule encoding a bioactive agent.

These and other aspects of the present invention will become evident upon reference to the following detailed description and attached drawings.

### BRIEF DESCRIPTION OF THE DRAWINGS

FIG. 1 is a schematic representation of an in situ bioreactor comprising a biocompatible matrix.

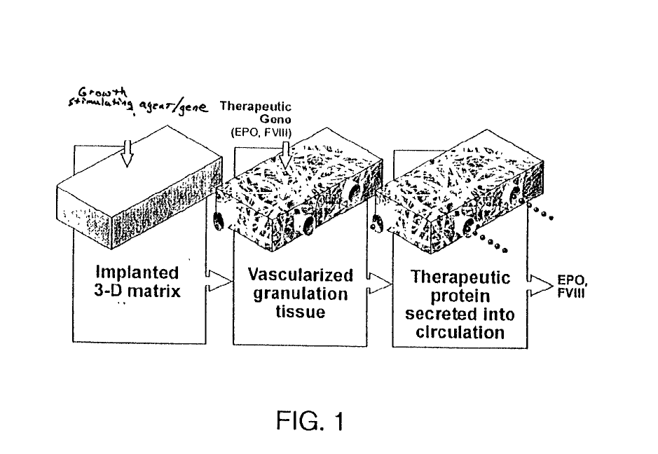

FIGS. 2A and 2B represent stained PVA sponge sections (2A) and a bar graph (2B) demonstrating that adenoviral delivery and expression of PDGF enhances new tissue formation within the sponge in vivo.

FIGS. 3A and 3B are bar graphs depicting the expression of a second transgene in the presence and absence of preconditioning with a growth factor. FIG. 3A demonstrates luciferase expression normalized for total protein following treatment with AdPDGF and no second gene, no first gene and AdLuc as the second gene, and AdPDGF as the first gene in combination with AdLuc as the second gene. FIG. 3B represents PDGFBB expression normalized for total protein under the same conditions of 3A.

### DETAILED DESCRIPTION OF THE INVENTION

Prior to setting forth the invention, it may be helpful to an understanding thereof to set forth definitions of certain terms that will be used hereinafter.

A “nucleic acid molecule”, as used herein, refers to a polynucleotide molecule in the form of a separate fragment or as a component of a larger nucleic acid construct, that has been separated from its source cell (including the chromosome it normally resides in) at least once in a substantially pure form. Nucleic acid molecules may be comprised of a wide variety of nucleotides, including DNA, RNA, nucleotide analogues, or some combination of these.

A “cell growth stimulating agent”, as used herein, refers to an RNA molecule (e.g., antisense or ribozymes), polypeptide, or peptide encoded ultimately by a deoxyribonucleic acid molecule which initiates or promotes cellular ingrowth/migration, survival/cellular maintenance, and/or proliferation either directly or indirectly.

A “cell growth stimulating small molecule”, refers to a cell growth stimulating agent that is a small molecule such as a steroidal hormone.

A “bioactive agent”, as used herein, refers to any polypeptide based substance, such as a therapeutic agent, enzyme, cytokine, receptor ligand, soluble receptor, antibody, hormone, etc., whose systemic availability over a period of time is desired or whose targeted delivery to a specific cell or tissue is desired to be effectuated through the circulation. The bioactive agent is preferably an agent that alleviates a deficiency or treats a disorder. The nucleic acids encoding the bioactive agents of the present invention may be derived from a variety of sources, synthetic and natural, and include recombinant nucleic acids.

A “biocompatible substance”, as used herein, refers to virtually any composition, including both biological (natural) and synthetic components so long as the substrate is capable of coexistence with living tissues or organisms without causing undue harm (e.g., artificial joint compositions, PLGA, etc.). A biocompatible substance may be a biocompatible matrix comprising an interior and exterior surface (e.g., a catheter, hollow fibers, pores, etc.), wherein the interior surface of the matrix is capable of supporting cellular ingrowth and, accordingly, is capable of being accessed by biological material (e.g., fluid and cells) of the surrounding environment. A biocompatible substance may also be any enclosure or compartment capable of being infiltrated by cells. Such an enclosure or compartment may be composed of a porous material or membrane through which living cells can migrate. Typically, the biocompatible substance will be a polymer. Such polymers should possess appropriate mechanical and physical properties; tissue, cell, and blood compatibility (minimum histotoxicity, noncarcinogenicity); appropriate aging properties in the implant site (stability or degradability); and should be capable of being made in a sterile fashion.

“Cellular infiltration”, as used herein, refers to cell migration in reference to a biocompatible substance. Cellular infiltration encompasses cell migration into and along the interior surface of a biocompatible substance such as a matrix. Cellular infiltration also includes cell migration across a permeable biocompatible substance. For example, cell infiltration describes cell migration across a permeable biocompatible membrane into a space or compartment enclosed by the membrane (see, e.g. Cell Encapsulation Technology and Therapeutics, (Kuhtreiber, Langer, Chick (eds.)), Birkhauser, Boston, 1999).

A “cell retention agent”, as used herein, refers to an agent that contributes directly or indirectly to cell retention within the interior of a biocompatible substance (e.g., device or matrix). Accordingly, such agents include extracellular matrix components, such as fibronectin, laminin, and the like. Also included are proteins that actively contribute to decreased migration such as macrophage migration inhibitory factor, which when placed within a matrix will act as “cell motel”, in that cells migrate in, but do not migrate out. Cell adhesion molecules (e.g., L-selectin) are also considered to be cell retention agents.

A “repair cell”, as used herein, refers to any cell which infiltrates a wound site during any stage of tissue repair, and may include, for example, macrophages, granulocytes, lymphocytes, fibroblasts, epithelial cells, monocytes, mast cells, megakaryocytes, endothelial cells, keratinocytes, mesenchymal cells, stem cells, and smooth muscle cells, etc.

Biocompatible Substances

An overarching principal of the present invention is that the biocompatible substance compositions are capable of supporting cellular ingrowth and harboring nucleic acid molecules and/or proteins that condition the environment (promote tissue growth or cellular proliferation/migration) for the uptake of nucleic acid molecules encoding bioactive agents. One of ordinary skill in the art can readily determine whether a particular substance is capable of cell ingrowth. At a minimum, the substance must have chambers, pores, or openings large enough for a cell to enter. Such ingrowth can be analyzed by several methodologies, including seeding the substance ex vivo and growing cells in culture on the substance and subsequently analyzing the substance for ingrowth. In addition, the substance may be implanted in an animal, such as mouse, for a time sufficient to induce ingrowth. The substance may then be removed and subjected to histological or microscopic analysis to determine the extent of cellular ingrowth. In particular embodiments, ingrowth is initiated via a wound response. While the wound itself may be iatrogenic (e.g., caused directly or indirectly by a physician) or due to pathology or traumatic injury, its source is unimportant as long as wound response is ongoing or initiated at the site of biocompatible substance placement.

A matrix is used herein as a protoypic example of a biocompatible substance having cell ingrowth capability. However, the current invention is not limited to matrices and thus, wherever the term matrix or matrices appears these terms should be read to include devices and other substances which allow for cell ingrowth are biocompatible and are capable of retaining a nucleic acid molecule within a defined internal area that is accessible to cells following in growth.

Matrices have been utilized for a number of years within the context of tissue engineering (see, e.g., Principles of Tissue Engineering (Lanza, Langer, and Chick (eds.)), 1997. However, the present invention utilizes such matrices within the novel context of nucleic acid delivery to cells to achieve systemic bioactive agent delivery. Accordingly, the present invention can utilize those matrix compositions and formulations which have demonstrated utility in tissue engineering. As the matrix need only support nucleic acid association (e.g., impregnation, adsorption, absorption, or chemical conjugation), and allow cell and vascular ingrowth, the type of matrix that may be used in the compositions, devices and methods of the invention is virtually limitless and may include both biological and synthetic matrices. In one particular aspect of the invention, compositions are prepared in which the nucleic acid encoding the therapeutic agent of interest is associated with or impregnated within a matrix containing a tissue-promoting gene to form a bi-gene device.

Matrices, within the context of the present invention, comprise features commonly associated with being biocompatible when administered to a mammalian host. Matrices may be formed from both natural or synthetic materials. The matrices may be non-biodegradable in instances where it is desirable to leave permanent structures or removable structures in the body, such as an implant; or biodegradable where the expression of the bioactive agent is required only for a short duration of time. The matrices may take the form of sponges, implants, tubes, telfa pads, band-aids, bandages, fibers, hollow fibers, sutures, pads, lyophilized components, gels, patches, powders, porous compositions, or nanoparticles. In addition, matrices can be designed to allow for sustained release of the nucleic acid over prolonged periods of time. In certain embodiments, the matrix of the present invention is flexible and elastic, and may be described as a semisolid scaffold that is permeable to substances such as cells, inorganic salts, aqueous fluids and dissolved gaseous agents including oxygen.

One of ordinary skill in the art understands that the choice of matrix material may differ according to the particular purpose for treatment and the site of matrix placement. Briefly, if a short term indication is to be treated, a biodegradable matrix may be more advantageous, while if longer term therapy is envisioned, a non-biodegradable matrix or coated device may be more appropriate. Matrices such as those described in U.S. Pat. Nos. 5,270,300; 5,514,378; 5,502,092 and in “Synthetic Biodegradable Polymer Scaffolds”, Atala and Mooney (eds.) Birkhauser, Boston, USA, 1997; Domb et al., Polymers for Advanced Technologies 3:279-292, 1992; “Biodegradable Polymers as Drug Delivery Systems”, Chasin and Langer (eds.) Vol. 45 of Drugs and the Pharmaceutical Sciences, M. Dekker, New York, 1990, are incorporated, herein by reference, in their entirety, may be employed. Physical and chemical characteristics, such as, e.g., biocompatibility, biodegradability, strength, rigidity, interface properties and even cosmetic appearance may be considered in choosing a matrix, as is well known to those of skill in the art. Appropriate matrices will both deliver the nucleic acid molecules and also act as an in situ scaffolding through which cells (e.g., repair cells) may migrate.

Where the matrices are to be maintained for extended periods of time, non-biodegradable matrices may be employed, such as sintered hydroxyapatite, bioglass, aluminates, other bioceramic materials and metal materials, particularly titanium. A suitable ceramic delivery system is that described in U.S. Pat. No. 4,596,574, incorporated herein by reference. The bioceramics may be altered in composition, such as in calcium-aluminate-phosphate; and they may be processed to modify particular physical and chemical characteristics, such as pore size, particle size, particle shape, and biodegradability. Polymeric matrices may also be employed, including acrylic ester polymers and lactic acid polymers, as disclosed in U.S. Pat. Nos. 4,521,909, and 4,563,489, respectively, each incorporated herein by reference. Particular examples of useful polymers are those of orthoesters, anhydrides, propylene-cofumarates, or a polymer of one or more cc-hydroxy carboxylic acid monomers, e.g., a-hydroxy auric acid (glycolic acid) and/or u-hydroxy propionic acid (lactic acid), nylon (polyamides), dacron (polyesters), polystyrene, polypropylene, polyacrylates, polyvinyl compounds (e.g., polyvinylchloride), polycarbonate (PVC), polyethylene (PE), polypropylene (PS), styrene-acrylonitrile copolymer (SAN), polytetrafluorethylene (PTFE, teflon), thermanox (TPX), and a variety of polyhydroxyalkanoates.

One aspect of the present invention is the use of the matrix connection with implants and interfaces (e.g., artificial joints), including implants themselves and functional parts of an implant, for example, surgical screws, pins, and the like. In preferred embodiments, it is contemplated that the metal surface or surfaces of an implant or a portion thereof, such as a titanium surface, will be coated with a material that has an affinity for nucleic acids, such as hydroxyl apatite, and then the coated-metal will be further coated with the gene or nucleic acid that one wishes to transfer. The available chemical groups of the absorptive material, such as hydroxyl apatite, may be readily manipulated to control its affinity for nucleic acids, as is known to those of skill in the art.

A biodegradable matrix is generally defined as one that is capable of being reabsorbed into the body. Potential biodegradable matrices for use in connection with the compositions, devices and methods of this invention include, for example, biodegradable and chemically defined calcium sulfate, tricalciumphosphate, hydroxyapatite, hyaluronic acids, polydihydropyrans, polyphosphazenes, poly(ortho esters), polycyanoacrylates, polyanhydrides, polydepsipeptides, aliphatic polyesters (e.g., polyglycolic acid, polylactic acid, copolymers thereof), matrices of purified proteins (e.g., collagen, fibrin, etc.), matrices of purified peptides, polysaccharides (e.g., cellulose, methyl cellulose, starch, chitin, etc.) and semi-purified extracellular matrix compositions.

Preferred biocompatible biodegradable matrices that may be used are well known in the art and include, by way of example and not limitation, polyesters such as polyglycolides, polylactides and polylactic polyglycolic acid copolymers (“PLGA”) (Langer and Folkman, Nature 263:797-800, 1976); hydrogels (e.g., cross-linked gelatin, poly(ethylene glycol monomethacrylate); polyaminotrizoles; polyethers such as polycaprolactone (“PCL”); polyanhydrides; polyalkyl cyanoacrylates such as n-butyl cyanoacrylate and isopropyl cyanoacrylate; hyaluronic acids; polyacrylamides; poly(orthoesters); polyphosphazenes; polypeptides; polyurethanes; and mixtures of such polymers).

It should be understood that virtually any polymer that is now known or that will be later developed that is suitable for the sustained or controlled release of nucleic acids and can be formed into a shape that allows cellular ingrowth may be employed in the present invention.

Four polymers that have been widely used in medical applications are poly(paradioxanone) (PDS), poly(lactic acid) (PLA), poly(glycolic acid) (PGA), and PLGA copolymers. Copolymerization enables modulation of the degradation time of the material. By changing the ratios of crystalline to amorphous polymers during polymerization, properties of the resulting material can be altered to suit the needs of the application. These polymers, including poly(lactide-co-glycolic) acid (PLGA), have been used as polymer composites for bone replacement as reported by Elgendy et al., Biomaterials 14:263-269, 1993. Substituted polyphosphazenes have been shown to support osteogenic cell ingrowth, as reported by Laurencin et al., J. Biom. Mater. Res. 27, 1993. Poly(organophosphazenes) are high molecular weight polymers containing a backbone of alternating phosphorus and nitrogen atoms. There are a wide variety of polyphosphazenes, each derived from the same precursor polymer, poly(dichlorophosphazene). The chlorine-substituted species can be modified by replacement of the chlorine atoms by different organic nucleophiles such as o-methylphenoxide along with amino acids. The physical and chemical properties of the polymer can be altered by adding various ratios of hydrolytic sensitive side chains such as ethyl glycinate, as described by Wade et al., in Organomet. Polym., Carraher, Sheats and Pitman, Jr., Eds., Academic Press, New York, pp. 283-288, 1978; and Allcock and Fuller, J. Am. Chem. Soc. 103:2250-2256, 1981. This will affect the degradation of the polymer as an implantable and biodegradable material as well as vary the support of cells.

PLA, PGA and PLA/PGA copolymers are particularly useful for forming the biodegradable matrices of the present invention. PLA polymers are usually prepared from the cyclic esters of lactic acids. Both L(+) and D(−) forms of lactic acid can be used to prepare the PLA polymers, as well as the optically inactive DL-lactic acid mixture of D(−) and L(+) lactic acids. Methods of preparing polylactides are well known in the art. PGA is the homopolymer of glycolic acid (hydroxyacetic acid). In the conversion of glycolic acid to poly(glycolic acid), glycolic acid is initially reacted with itself to form the cyclic ester glycolide, which in the presence of heat and a catalyst is converted to a high molecular weight linear-chain polymer.

The erosion of the matrix is related to the molecular weights of PLA, PGA or PLA/PGA. The higher molecular weights, average 90,000 or higher, result in polymer matrices which retain their structural integrity for longer periods of time; while lower molecular weights, weight average molecular weights of 30,000 or less, result in both slower release and shorter matrix lives. Poly(lactide-co-glycolide) (50:50), degrades in about six weeks following implantation.

All polymers for use in the matrix must meet the mechanical and biochemical parameters necessary to provide adequate support for the cells with subsequent growth and proliferation. The polymers can be characterized with respect to mechanical properties such as tensile strength using an Instron tester, for polymer molecular weight by gel permeation chromatography (GPC), glass transition temperature by differential scanning calorimetry (DSC) and bond structure by infrared (IR) spectroscopy, with respect to toxicology by initial screening tests involving Ames assays and in vitro teratogenicity assays, and implantation studies in animals for immunogenicity, inflammation, release and degradation studies.

These polymers are particularly useful in forming fibrous or sponge type matrices for implantation. In preferred embodiments, the biocompatible biodegradable polymer is a copolymer of glycolic acid and lactic acid (“PLGA”) having a proportion between the lactic acid/glycolic acid units ranging from about 100/0 to about 25/75. The average molecular weight (“MW”) of the polymer will typically range from about 6,000 to 700,000 and preferably from about 30,000 to 120,000, as determined by gel-permeation chromatography using commercially available polystyrene of standard molecular weight, and have an intrinsic viscosity ranging from 0.5 to 10.5.

As noted above, the length of the period of continuous sustained or controlled release of nucleic acids from the matrix according to the invention will depend in large part on the MW of the polymer and the composition ratio of lactic acid/glycolic acid. Generally, a higher ratio of lactic acid/glycolic acid, such as for example 75/25, will provide for a longer period of controlled of sustained release of the nucleic acids, whereas a lower ratio of lactic acid/glycolic acid will provide for more rapid release of the nucleic acids. Preferably, the lactic acid/glycolic acid ratio is 50/50.

The length of time of sustained or controlled release is also dependent on the MW of the polymer. Generally, a higher MW or higher crosslinked polymer will provide for a longer period of controlled or sustained release. In the case of preparing, for example, matrices providing controlled or sustained release for about three months, when the composition ratio of lactic acid/glycolic acid is 100/0, the preferable average MW of polymer ranges from about 7,000 to 25,000; when 90/10, from about 6,000 to 30,000; and when 80/20, from about 12,000 to 30,000.

Another particular example of a suitable material is fibrous collagen, which may be lyophilized following extraction and partial purification from tissue and then sterilized. Matrices may also be prepared from tendon or dermal collagen, as may be obtained from a variety of commercial sources, such as, e.g., Sigma and Collagen Corporation. Collagen matrices may also be prepared as described in U.S. Pat. Nos. 4,394,370 and 4,975,527, each incorporated herein by reference.

In addition, lattices made of collagen and glycosaminoglycan (GAG) such as that described in Yannas & Burke, U.S. Pat. No. 4,505,266, may be used in the practice of the invention. The collagen/GAG matrix may effectively serve as a support or “scaffolding” structure into which cells may migrate. Collagen matrices, such as those disclosed in Bell, U.S. Pat. No. 4,485,097, may also be used as a matrix material.

The various collagenous materials may also be in the form of mineralized collagen. For example, the fibrous collagen implant material termed UltraFiber™, as may be obtained from Norian Corp., (Mountain View, Calif.) may be used for formation of matrices. U.S. Pat. No. 5,231,169, incorporated herein by reference, describes the preparation of mineralized collagen through the formation of calcium phosphate mineral under mild agitation in situ in the presence of dispersed collagen fibrils. Such a formulation may be employed in the context of delivering a nucleic acid molecule to a tissue site. Mineralized collagen, as with any matrix composition, may be employed, for example, as part of kit for producing an in situ bioreactor for use in vivo.

A variety of different forms of collagen have been identified and each of these collagens may be used in the practice of the invention. For example, collagen may be purified from hyaline cartilage, as isolated from diarthrodial joints or growth plates. Atecollagen may also be used according to the invention. Type II collagen purified from hyaline cartilage is commercially available and may be purchased from, e.g., Sigma Chemical Company, St. Louis. Type I collagen from rat tail tendon may be purchased from, e.g., Collagen Corporation or Cohesion Technologies. Any form of recombinant collagen may also be employed, as may be obtained from a collagen-expressing recombinant host cell, including bacterial yeast, mammalian, and insect cells. When using collagen as a matrix material it may be advantageous to remove what is referred to as the “telopeptide” which is located at the end of the collagen molecule. The resulting atelocollagen is known to decrease the inflammatory response toward collagen.

The collagen used in the invention may, if desired be supplemented with additional minerals, such as calcium, e.g., in the form of calcium phosphate. Both native and recombinant type collagen may be supplemented by admixing, absorbing, or otherwise associating with, additional minerals in this manner.

In one embodiment the matrix comprises a hydrogel. The term “hydrogel”, as used herein, refers to a three dimensional network of cross-linked hydrophilic polymers. The network is in the form of a gel substantially composed of water, preferably but not limited to gels being greater than 90% water. Cross-linked hydrogels can also be considered solids because they do not flow or deform without appreciable applied shear stress. Compositions that form hydrogels generally fall into three classes. The first class carries a net negative charge and is typified by alginate. The second class carries a net positive charge and is typified by extracellular matrix components such as collagen and laminin. Examples of commercially available extracellular matrix components include MATRIGEL™ (Becton Dickinson Labware, N.J.) and VITROGEN™ (Collagen Corp. or Cohesion Technologies, Calif.). The third class is net neutral in charge. An example of a net neutral hydrogel is highly crosslinked polyethylene oxide, or polyvinylalcohol.

Polymers that can form ionic hydrogels which are malleable can also be used to support the cells. Injecting a suspension of nucleic acid molecules, a gene delivery device, or cells into a polymer solution may be performed to improve the reproducibility and uniformity throughout a matrix, to protect the components from shear forces or pressure induced necrosis (for cells), or to aid in defining the spatial location. The injectable polymer may also be utilized to deliver bi-gene devices or in situ bioreactors and promote the formation of new tissue without the use of any other matrix. In a preferred embodiment, the hydrogel is produced by cross-linking the ionic salt of a polymer with ions. The strength of the hydrogel increases with either increasing concentrations of ions or polymer. The polymer solution is mixed with the appropriate components, such as nucleic acid molecules, gene delivery compositions, polypeptides, etc. to form a suspension, which is then injected directly into a patient prior to hardening of the suspension. The suspension subsequently hardens over a short period of time due to the presence in vivo of physiological concentrations of ions such as calcium, as is the case where the polymer is a polysaccharide such as alginate.

A “hydrogel”, as used herein is a matrix composition, and is a substance formed when an organic polymer (natural or synthetic) is cross-linked via covalent, ionic, or hydrogen bonds to create a three-dimensional open-lattice structure which entraps water molecules to form a gel. Examples of materials which can be used to form such a hydrogel include polysaccharides such as alginate, polyphosphazenes, and polyacrylates such as hydroxyethyl methacrylate (HEMA), which are crosslinked ionically, or block copolymers such as PLURONICS™ or TETRONICS™, polyethylene oxide-polypropylene glycol block copolymers which are crosslinked by temperature or pH, respectively. Other materials include proteins such as fibrin, polymers such as polyvinylpyrrolidone, hyaluronic acid and collagen.

In general, these polymers are at least partially soluble in aqueous solutions, such as water, buffered salt solutions, or aqueous alcohol solutions, that have charged side groups, or a monovalent ionic salt thereof. Examples of polymers with acidic side groups that can be reacted with cations are poly(phosphazenes), poly(acrylic acids), poly(methacrylic acids), copolymers of acrylic acid and methacrylic acid, poly(vinyl acetate), and sulfonated polymers, such as sulfonated polystyrene. Copolymers having acidic side groups formed by reaction of acrylic or methacrylic acid and vinyl ether monomers or polymers can also be used. Examples of acidic groups are carboxylic acid groups, sulfonic acid groups, halogenated (preferably fluorinated) alcohol groups, phenolic OH groups, and acidic OH groups. Examples of polymers with basic side groups that can be reacted with anions are poly(vinyl amines), poly(vinyl pyridine), poly(vinyl imidazole), and some imino substituted polyphosphazenes. The ammonium or quaternary salt of the polymers can also be formed from the backbone nitrogens or pendant imino groups. Examples of basic side groups are amino and imino groups.

Alginate can be ionically cross-linked with divalent cations, in water, at room temperature, to form a hydrogel matrix. Due to these mild conditions, alginate has been the most commonly used polymer for cell encapsulation, as described, for example, in U.S. Pat. No. 4,352,883 to Lim. In the Lim process, an aqueous solution containing the biological materials to be encapsulated is suspended in a solution of a water soluble polymer, the suspension is formed into droplets which are configured into discrete microcapsules by contact with multivalent cations, then the surface of the microcapsules is crosslinked with polyamino acids to form a semipermeable membrane around the encapsulated materials.

The polyphosphazenes suitable for cross-linking have a majority of side chain groups which are acidic and capable of forming salt bridges with di- or trivalent cations. Examples of preferred acidic side groups are carboxylic acid groups and sulfonic acid groups. Hydrolytically stable polyphosphazenes are formed of monomers having carboxylic acid side groups that are crosslinked by divalent or trivalent cations such as Ca²⁺ or Al³⁺. Hydrogel forming polymers may be synthesized to degrade by hydrolysis by incorporating monomers having imidazole, amino acid ester, or glycerol side groups. Biodegradable polyphosphazenes have at least two differing types of side chains, acidic side groups capable of forming salt bridges with multivalent cations, and side groups that hydrolyze under in vivo conditions, e.g., imidazole groups, amino acid esters, glycerol and glucosyl.

The water soluble polymer with charged side groups is crosslinked by reacting the polymer with an aqueous solution containing multivalent ions of the opposite charge, either multivalent cations if the polymer has acidic side groups or multivalent anions if the polymer has basic side groups. The preferred cations for cross-linking of the polymers with acidic side groups to form a hydrogel are divalent and trivalent cations such as copper, calcium, aluminum, magnesium, strontium, barium, and tin, although di-, tri- or tetra-functional organic cations such as alkylammonium salts, can also be used. Aqueous solutions of the salts of these cations are added to the polymers to form soft, highly swollen hydrogels and membranes. The higher the concentration of cation, or the higher the valence, the greater the degree of cross-linking of the polymer. Concentrations from as low as 0.005 M have been demonstrated to cross-link the polymer. Higher concentrations are limited by the solubility of the salt. The preferred anions for cross-linking of the polymers to form a hydrogel are divalent and trivalent anions such as low molecular weight dicarboxylic acids, for example, terepthalic acid, sulfate ions and carbonate ions. Aqueous solutions of the salts of these anions are added to the polymers to form soft, highly swollen hydrogels and membranes, as described with respect to cations.

A variety of polycations can be used to complex and thereby stabilize the polymer hydrogel into a lattice. Examples of materials that can be used include polymers having basic reactive groups such as amine or imine groups, having a preferred molecular weight between 3,000 and 100,000, such as polyethylenimine and polylysine. These are commercially available. One polycation is poly(L-lysine), examples of synthetic polyamines are: polyethyleneimine, poly(vinylamine), and poly(allyl amine). There are also natural polycations such as the polysaccharide, chitosan. Polyanions that can be used by reaction with basic surface groups on the polymer hydrogel include polymers and copolymers of acrylic acid, methacrylic acid, and other derivatives of acrylic acid, polymers with pendant SO₃H groups such as sulfonated polystyrene, and polystyrene with carboxylic acid groups.

Accordingly, virtually any gel can be used in the practice of the present invention. The materials which can be used to form such gels include but are not limited to: carbohydrates such as cellulosics, methylcellulose, starch and modified starch, agarose, gum arabic, ghatti, karay, tragacanth, guar, locust bean gum, tamarind, carageenan, alginate, xanthan, chickle, collagen, polyacrylamide, polysiloxanes (polyanhydrides, e.g., malic anhydride copolymers, polyacrylates, e.g., hydroxyethylpolymethycrylate polymethylmethacrylate, polyethylethacrylate polymethacrylate, ethylenevinylacetate copolymers, ethylenevinylalcohol copolymers, polyorthoesters, ε-caprolactones, amino acid polymers such as gelled albumin, amino acid polymers and copolymers and gelatins, and other organic or inorganic polymers which may be mixed with liposomes in vitro.

In certain embodiments, the neo-organoid or bi-gene device should comprise a matrix and have sufficient surface area and exposure to nutrients such that cellular ingrowth and differentiation can occur prior or concurrent to the ingrowth of blood vessels. After implantation, the configuration must allow for diffusion of nutrients and waste products and for continued blood vessel ingrowth as cell proliferation occurs. The organization of the growing tissue may be regulated by the microstructure of the matrix: Specific pore sizes and structures may be utilized to control the pattern and extent of fibrovascular tissue ingrowth from the host. Accordingly, if cells are seeded in the matrix before implantation the organization of the seeded cells may also be guided. The surface geometry and chemistry of the matrix may be regulated to control the adhesion (e.g., by extracellular matrix proteins such as laminin, collagen, thrombospondin, collagen, elastin, fibronectin, tenascin, entactin, vitronectin, and the like), organization, and function of seeded cells or ingrowing host cells.

In certain embodiments, the matrix is formed of polymers having a fibrous structure which has sufficient interstitial spacing typically in the range of 100 to 300 microns (see, Friedlander and Goldberg, Bone and Cartilage Allografts, Park Ridge: American Academy of Orthopedic Surgeons, 1991; Jarcho, Clin Orthopedics and Related Research 157:259-278, 1981. As used herein, “fibrous” includes one or more fibers that is entwined with itself, multiple fibers in a woven or non-woven mesh, and sponge like devices.

In certain various embodiments of the invention, the matrix may comprise or be modified, e.g., coated or impregnated, prior to implantation with certain substances to enhance the attachment and growth of cells on the matrix in vivo. These substances include, but are not limited to, bioactive agents such cellular growth factors (e.g., TGF-β, FGF, etc.), substances that stimulate chondrogenesis (e.g., BMPs that stimulate cartilage formation such as BMP-2, BMP-12 and BMP-13), factors that stimulate migration of cells to the matrix, factors that stimulate matrix deposition, substances or factors that stimulate angiogenesis (e.g., anti-Thrombospondin 2 antibodies), anti-inflammatories (e.g., non-steroidal anti-inflammatories), immunosuppressants (e.g., cyclosporins), as well as other proteins, such as collagens, elastic fibers, reticular fibers, glycoproteins or glycosaminoglycans, such as heparin sulfate, chondroitin-4-sulfate, chondroitin-6-sulfate, dermatan sulfate, keratin sulfate, etc. The bioactive agent may also be a cell retention agent, such as laminin, fibronectin or the like to adhere cells to the matrix, or may be an active inhibitor of cellular migration such as macrophage migration inhibitory factor (MIF). One of ordinary skill in the art will readily recognize that such agents may either be in the form of polypeptides or in the form of nucleic acid molecules encoding such polypeptides, such that upon implantation such nucleic acid molecules are taken up by the migrating cells and expressed.

Bioreactor

The present invention relates to a method of in vivo, sustained gene therapy wherein one or more in situ bioreactors (or neo-organoids) express systemically available bioactive agents. One method of the invention involves implanting or placing into a tissue site a biocompatible substance capable of cellular ingrowth (e.g., device, matrix, semi-permeable membrane with a matrix or liquid interior, etc.) containing at least a first nucleic acid or polypeptide molecule encoding a cell growth promoting agent and either concomitantly or subsequently providing at least a second nucleic acid molecule encoding a bioactive agent capable of functioning systemically. The invention is based on the discovery that the proliferation and migration of repair cells to sites of tissue injury can be enhanced by local delivery of a growth promoting nucleic acid or polypeptide and that infiltrating repair cells are capable of uptaking exogenous nucleic acids and polypeptides. Implantation of a matrix which supports tissue growth and vascularization provides a means to deliver nucleic acids and polypeptides with growth promoting and/or other bioactive properties which may have therapeutic effects. In addition, bioactive molecules can pass into the bloodstream via vessels associated with the bioreactor, thus providing a means of systemic delivery.

Wound healing is understood to be a coordinated sequence of events that includes (a) tissue disruption and loss of normal tissue architecture; (b) cell necrosis, hemorrhage, and hemostasis (clot formation); (c) infiltration of segmented and mononuclear inflammatory cells, followed by vascular congestion and tissue edema; (d) mononuclear cell (macrophage) directed clot dissolution and concomitant elimination of damaged tissue; (e) formation of granulation tissue, which includes, for example, fibroblasts, endothelial cells and extracellular matrix (ECM). This sequence of cellular events has been observed in wounds from all tissues and organs generated in a large number of mammalian species (Gailet et al., Curr. Opin. Cell. Biol. 6:717-725, 1994). Therefore, the cellular sequence described above appears to be a universal aspect of the repair of all mammalian tissues.

As an initial step in generating a bioreactor or neo-organoid, the composition, devices and methods of the present invention are designed to efficiently transfer one or more different nucleic acid or polypeptide molecules encoding cell growth promoting agents to matrix infiltrating cells such as repair cells, for example. During the healing process, repair cells actively migrate and proliferate to the wound site, a process which makes the cells extremely competent to take up and express exogenous nucleic acid molecules. The present invention involves the administration of a matrix comprising at least one cell growth promoting agent. In one aspect, the cell growth promoting agent is encoded by a nucleic acid molecule. The expressed cell growth promoting agent may be in the form of translational products (e.g., growth factors) or transcriptional products (e.g., antisense nucleic acids or ribozymes). As noted above, the tissue damage (i.e., a wound) may be iatrogenic, traumatic injury or due to pathology.

As the proliferating cells migrate into and contact the matrix, they take up and express the nucleic acid of interest, thereby promoting an additional influx of repair cells such as fibroblasts and endothelial cells. The recombinant repair cells may produce, for example, growth factors or cytokines that will stimulate targeted effector cells to express cognate cell surface receptors, thereby inducing an amplification of the cascade of physiological events normally associated with the wound healing process.

Alternatively, the repair cells may take up and express nucleic acid molecules encoding proteins that inhibit the activity of antagonists of the wound healing process. The nucleic acid molecules may also encode for antisense or ribozyme RNA molecules that may be used to inhibit translation of mRNAs encoding inflammatory proteins or other factors that inhibit wound healing or cause excessive fibrosis.

In addition, the recombinant repair cells may induce granulation tissue formation within the matrix, including fibroblast proliferation, production of extracellular matrix components (e.g., collagen), and angiogenesis. Thus, the bioreactor within and surrounding the matrix provides an in vivo supply of cells that are localized in a vascularized compartment and are conditioned for the uptake of exogenous nucleic acid molecules or transgenes.

Conditioning and Implantation

Implantable biocompatible substances (e.g., device, matrix, semi-permeable membrane with a matrix or liquid interior, etc.) may contain growth promoting nucleic acids and/or polypeptides prior to implantation, or such nucleic acids and/or polypeptides may be provided to the substance following implantation. In certain embodiments, the matrix may be conditioned prior to implantation. The matrix or implant material is contacted with a nucleic acid and/or polypeptide biological agent in a suitable buffer solution. Pharmaceutical grade buffers suitable for recombinant nucleic acid molecules and proteins are known in the art. The amount of nucleic acid molecules or polypeptide and the length of contact time required for incorporation of the DNA or polypeptide into the matrix will depend on the structure and composition of the particular matrix employed and the implant size. Appropriate amounts of nucleic acid or polypeptide and contact times can be readily determined by one of ordinary skill in the art without undue experimentation. See also, U.S. Pat. Nos. 5,942,496 and 5,962,427 which are incorporated in their entirety and which describe matrix preparation and implantation within the context of localized delivery of a therapeutic agent.

Alternatively, biological agents such as nucleic acid molecules and polypeptides may be encapsulated within a matrix of synthetic polymers, such as, for example, block copolymers of polylactic-polyglycolic acid (See Langer and Folkman, Nature 263:297-800, 1976, which is incorporated by reference). The amount of biological agent to be encapsulated can be readily determined by one of ordinary skill in the art.

Biological and medical factors to be weighed in determining the appropriate amount of nucleic acid and/or polypeptide applied to or incorporated within the matrix may include, for example, the particular nucleic acid, the particular promoter driving expression, the type of matrix employed, the site of the wound, the host's age, sex and diet, the medical condition being treated, the severity of the medical condition, and any other clinical factors that may effect wound healing such as serum levels of various factors and hormones and the predicted immune response.

In certain embodiments, compositions of both biological and synthetic matrices and nucleic acid molecules may be lyophilized together to form a dry pharmaceutical powder. The matrix may be rehydrated prior to implantation in the body, or alternatively, the matrix may become naturally rehydrated when placed in the body.

Bioreactors may include medical devices such as, for example, stents, catheters, synthetic joints, implants and sutures. In certain embodiments of the invention, such medical devices and other matrices may be coated with nucleic acids and/or polypeptides using conventional coating techniques as are well known in the art. Such methods include, by way of example and not limitation, dipping the device in the nucleic acid and/or polypeptide, brushing the device with the nucleic acid and/or polypeptide or spraying the device with aerosol compositions comprising nucleic acids and/or polypeptides. Matrices may be dried, either at room temperature or in a drying oven, optionally at reduced pressure and temperature.

The bioreactor matrix can be transferred to the host patient by a variety of techniques. In one embodiment of the invention, the matrix can be transferred directly to the site of a naturally occurring wound or an iatrogenic injury. In certain embodiments, matrices may be surgically placed in a wound made in an organ, implanted subcutaneously, or topically administered to a skin wound. Matrices may also be implanted via grafting, injection, catheterization, laproscopic surgical procedures or arthroscopic surgery.

In instances where the matrices are injected, the matrices may be drawn into a syringe and injected into a patient at the site of a naturally occurring or iatrogenic wound produced by, or resulting from, a surgical procedure. Single or multiple injections may be performed at one or more wound sites. The amount of matrices needed to produce the desired therapeutic effect is variable depending on biological and medical factors including, but not limited to, the specific gene or polypeptide expressed, the promoter driving gene expression, the particular disease and its severity, the age, weight, and medical condition of the patient, and the location of the wound site. The amount of matrices to implant can be readily determined without undue experimentation by measuring the serum levels of bioactive molecules expressed by bioreactors supported by matrices of different size or composition. Additionally, or alternatively, the therapeutic effect of different amounts of implanted matrix can be determined by examining clinical disease indicators.

The present invention discloses methods, compositions and devices using both permanent and temporary bioreactor organoids. Permanent bioreactors which allow for the continuous administration of bioactive nucleic acids or polypeptides are particularly useful for treating chronic or long-term diseases or related medical conditions. The continued function of permanent bioreactors can be monitored by any appropriate biological assay available including, but not limited to, measuring the blood serum levels of proteins or polypeptides produced by the bioreactor, measuring the levels of circulating antibodies directed against bioreactor produced proteins or polypeptides, or by determining mRNA production by the bioreactor in a biopsy sample obtained from tissue surrounding or infiltrating the bioreactor. Efficacy of the bioreactor will be determined by determining the therapeutic effect of the implanted bioreactor on the host patient. Clinical disease indicators and symptoms can be monitored by means available to one skilled in the art.

Permanent or temporary bioreactors may be recharged by applying additional nucleic acids or polypeptides to the underlying matrix and/or surrounding or infiltrating cells. The additional nucleic acids or polypeptides may be the same as those originally applied or different. For example, if an implanted bioreactor initially fails to produce or ceases producing a sufficient amount of therapeutic products, growth promoting nucleic acids and/or polypeptides or nucleic acids encoding bioactive agents may be reapplied to the bioreactor. Reapplication may occur by injection or by a surgical procedure. In certain instances, it is advantageous to periodically reapply nucleic acids and/or polypeptides to the bioreactor. In some instances, it is advantageous to provide an agent to the bioreactor which will re-stimulate an immune response or a wound healing response. This agent may, for example, have a cytotoxic effect on cells within or associated with the bioreactor, thus producing a secondary wound healing response. Alternatively, this agent may reactivate cells (e.g., repair cells) within or associated with the bioreactor.

Implanted matrices supporting the bioreactors may be either biodegradable or non-biodegradable. It is also within the scope of the current invention to remove a bioreactor from a host patient. Degradation or removal of a bioreactor may be indicated when the disease or associated medical condition being treated using the bioreactor is short-lived or cured. Additionally, removal of the bioreactor is appropriate if intolerable side effects are associated with permanent implantation or when examination of the bioreactor is desired for research or diagnostic purposes.

Since the method of the invention is based on the natural migration and proliferation of cells into a wound site and into the matrix located at the wound site, followed by uptake of nucleic acids or polypeptides, it is understood that the matrices will be transferred into a tissue site in the body where the wound healing process has been induced. The local tissue damage must be sufficient to induce the wound healing response. Furthermore, in order to allow systemic delivery of the expressed gene products produced by the bioreactor, the matrices must be implanted into a site capable of supporting vascularization of the bioreactor.

Nucleic Acids and Polypeptides

The present methods and compositions may employ a variety of different types of nucleic acid and polypeptide molecules. DNA molecules may include genomic DNA, cDNAs, single-stranded DNA, double-stranded DNA, oligonucleotides and Z-DNA. RNA molecules may include ribosomal RNA, messenger RNA, mitochondrial RNA, sense RNA, and antisense RNA. Polypeptide molecules may include proteins, fragments or variants thereof, antibodies, and ribozymes.

In a preferred embodiment, the bioreactor and methods of use thereof comprise a first nucleic acid molecule encoding a growth factor which stimulates migration and infiltration of repair cells into the matrix associated with the wound site. Alternatively, the first bioactive agent delivered may be a polypeptide, steroid, or RNA molecule. A large number of growth factors have been described, and the current invention includes these and any functional equivalents. Functional equivalents encompass any nucleic acid, polypeptide or hormone which induces proliferation of cells associated with the wound repair process. Such cells include, but are not limited to, fibroblasts, macrophages, monocytes, stem cells, leukocytes, lymphocytes, keratinocytes, and vascular cells including smooth muscle cells and endothelial cells. Growth factors and functional equivalents can be identified by routine methods available in the art. Polypeptide growth factors may include, for example, transforming growth factors-beta (TGF-β), fibroblast growth factors (FGF), keratinocyte growth factors (KGF), platelet derived growth factors (PDGF), insulin-like growth factors (IGF), bone morphogenic factors (BMP), vascular endothelial growth factors (VEGF), hepatocyte growth factors (HGF), epidermal growth factors (EGF), interleukins (IL), macrophage-colony stimulating factor (M-CSF) and granulocyte macrophage-colony stimulating factor (GM-CSF). PDGF includes, but is not necessarily limited to, PDGF-A homodimers, PDGF-B homodimers, and heterodimers of PDGF-A and PDGF-B. TGF-β includes any and all isoforms, including TGF-β1, TGF-β2 and TGF-β3. In specific embodiments, the polypeptide growth factor is, for example, PDGF-AA, PDGF-BB, PDGF-AB, HGF, FGF-1, FGF-2, FGF-3, FGF-4, FGF-5, FGF-6, FGF-7, FGF-8, FGF-9, FGF-10, FGF-11, FGF-12, FGF-13, FGF-14, FGF-15, FGF-16, FGF-17, FGF-18, FGF-19, FGF-20, FGF-21, KGF-2, TGF-β1, TGF-β2, or TGF-β3. In certain embodiments, the growth factor may be a mutated FGF-2. Mutagenesis of cysteine 78 of FGF-2 to serine ([C78S]FGF) or cysteine 96 to serine ([C96S]FGF) produced two mutants that retain virtually complete proliferative activity of native FGF-2. The construction and biological activity of FGF-1 with cysteine substitutions of one, two or all three cysteines has been disclosed (U.S. Pat. No. 5,223,483). Thus, any of the cysteines may be mutated and FGF-2 will still bind and internalize.

Cell growth promoting agents may function either directly or indirectly. Thus, cell growth promoting agents include, for example, transcription factors, chemotactic factors, hormones, cell surface receptors, ligands, angiogenic factors, anti-apoptotic agents, antibodies and extracellular matrix molecules. Transcription factors may activate genes encoding secreted growth factors or cell surface receptors. Examples of transcription factors which may be used include NF-κB, myc, AP-1, AP-2, p53, Sp1, NFAT, E2F, DP1, CBP, C/EBP, and nuclear hormone receptors. Chemotactic factors and extracellular matrix molecules attract repair cells to the wound site-associated matrix and provide physical support for migrating cells, respectively. Antibodies may activate immune cells and induce proliferation by engaging signal transduction pathways directing cell growth. Antibodies directed against growth factor receptors may induce proliferation by activating signal transduction pathways identical or similar to those activated by a physiological ligand. Angiogenic factors support proliferation of cells within the bioreactor matrix by inducing vascularization. Anti-apoptotic molecules reduce or block apoptosis of cells associated with the bioreactor matrix. Thus, anti-apoptotic molecules indirectly support tissue growth within and surrounding the bioreactor by facilitating the accumulation of living cells. A number of such molecules are known in the art and a variety are detailed in “When Cells Die” (Lockshin, Zakeri, and Tilly (Eds.)), Wiley-Liss, Inc., 1998). In certain embodiments, the anti-apoptotic agents include, for example, Bcl-2, Bcl-xL, or A20.

Growth factors within the scope of the current invention also include antagonists of growth inhibitors such as antisense molecules and ribozymes targeted to differentiation factors, cell cycle inhibitors and apoptotic molecules, thereby preventing senescence. Inactivating antibodies directed against growth inhibitors are included within the scope of the invention.

Ribozymes are trans-cleaving catalytic RNA molecules possessing endoribonuclease activity. Ribozymes are specifically designed for a particular target nucleotide sequence. Ribozymes are engineered to cleave an RNA species site-specifically in the background of cellular RNA. The cleavage event renders the mRNA unstable and prevents protein expression. Preparation and usage of ribozymes is well known to the art (see Usman et al., Current Opin. Struct. Biol. 6:527-533, 1996; Long et al., FASEB J. 7:25, 1993; Symons, Ann. Rev. Biochem. 61:641, 1992 and U.S. Pat. No. 5,254,678). Knowledge of the nucleotide sequence of the target ribonucleic acid molecule allows construction of an effective ribozyme.

Antisense nucleic acids are designed to specifically bind to RNA, resulting in the formation of RNA-DNA or RNA-RNA hybrids and an arrest in DNA replication, transcription or messenger RNA translation. Antisense polynucleotides based on a selected sequence can specifically interfere with expression of the corresponding gene. Antisense polynucleotides are typically generated within the cell by expression from antisense constructs that contain the antisense strand as the transcribed strand. Antisense production and uses thereof are discussed extensively in the literature and are widely known and available to one skilled in the art.

The present invention also encompasses the use of mutant and variant forms of genes or polypeptides, including dominant negative mutants. Dominant negative mutations are readily generated for a variety of proteins, including those which are active in homo- or heteromeric complexes. A mutant polypeptide will interact with wild-type binding partners, including, for example, cell surface receptors or ligands thereof, and form a non-functional multimer or a multimer with altered, decreased or enhanced function. Thus, a preferred mutation is in a substrate binding domain, a catalytic domain, or a cellular localization domain. Preferably, the mutant polypeptide will be overproduced compared to wild type expression. Point mutations and deletions are made which have such an effect. In addition, fusion of different polypeptides of various lengths to the terminus of a protein can yield dominant negative mutants (see Herskowitz, Nature 329:219-222, 1987).

The nucleic acids and polypeptides used in the invention include those possessing naturally occurring nucleotide and amino acid sequences and functional variants thereof. Polypeptides can be encoded by nucleic acids that, by virtue of the degeneracy of the genetic code, are not identical in sequence to naturally occurring genes, cDNAs, or mRNAs. Variants and mutants can include amino acid substitutions, additions or deletions. Amino acid substitutions can be conservative amino acid substitutions or substitutions to eliminate non-essential amino acids, such as to alter a glycosylation site, a phosphorylation site or an acetylation site, or to minimize misfolding by substitution or deletion of one or more cysteine residues. Conservative amino acid substitutions are those that preserve the general characteristics of the polypeptide, including charge, hydrophobicity/hydrophilicity, and/or steric bulk of the amino acid substituted.

The use of antibodies to induce cell proliferation within and surrounding the bioreactor is within the scope of the current invention. Antibodies may be monoclonal, polyclonal or single-chain antibodies. Antibodies can stimulate an inflammatory response and induce cell proliferation by interacting with cell surface receptors on immune cells and other repair cells. Activation of the inflammatory response initiates a cascade of cellular events, including secretion of cytokines and other inflammatory mediators which stimulate cell proliferation and migration to the wound site. Antibodies may be produced according to methods well known in the art. Antibodies which promote cell growth have been described in the literature, and others may be identified, for example in tissue culture experiments, without undue experimentation. In certain embodiments of the invention, antibodies utilized may be humanized or rendered less immunogenic to reduce associated immune responses.

The bioreactor may further comprise a cell retention agent. Nucleic acids encoding cell retention agents or polypeptide cell retention agents may be provided prior to implantation or post-implantation. Cell retention agents are provided to promote cellular infiltration of the bioreactor matrix by inhibiting cell migration from the bioreactor. One preferred example of a cell retention agent is macrophage migration inhibitory factor (MIF). MIF has been extensively characterized in the literature and has been shown to effect macrophage adherence and promote cytokine production, amongst other activities (Galat et al., Fed. Eur. Biochem. Soc. 319:233-236, 1993; Wistow et al., Proc. Natl. Acad. Sci. USA 90:1272-1275, 1993; Weiser et al., Proc. Natl. Acad. Sci. USA 86:7522-7526, 1989; Bernhagen et al., Nature 365:756-759, 1993; Blocki et al., Protein Science 2:2095-2102, 1993; Blocki et al., Nature 360:269-270, 1992; and Weiser et al., J. Immunol. 147:2006-2011, 1991). Extracellular matrix molecules such as fibronectin, collagen, laminin, vitronectin and thrombospondin may also function as cell retention agents within the bioreactor.

The nucleic acid encoding the bioactive agent to be delivered may include for example, genomic DNA, cDNA, single-stranded DNA, double-stranded DNA, or mRNA. In a preferred embodiment of the invention, the nucleic acid will encode a therapeutic polypeptide. In particular embodiments of the invention, the therapeutic nucleic acid will encode a soluble, secreted polypeptide suitable for systemic delivery via the host patient's bloodstream. The polypeptide may also have local effects which contribute to cell growth within the bioreactor, promote wound healing, alter a host's immune response and/or provide localized therapeutic treatment for the patient's disease or medical condition.

The bioactive nucleic acids and/or polypeptides of the bioreactor may encode any of a variety of polypeptides depending on the envisioned therapeutic use. Such proteins may include, for example, hormones, growth and differentiation factors, cytokines, hormones, cytokine inhibitors, clotting factors or thrombolytic proteins, enzymes for lipid storage, fibrinolytic agents, anticoagulants, anti-inflammatory agents, tumorocidal proteins, angiogenic factors, interferons or antibodies. Examples of such proteins include, but are not limited to, factor VIII, factor IX, factor VII, erythropoietin, growth hormone, insulin, atrial natriuretic peptide (ANP), luteinizing hormone, follicle-stimulating hormone, releasing hormone, follitropin, parathyroid hormone, activins, inhibins, adenosine deaminase, factor XIII, Protein C, Protein S, an interleukin, an interferon, insulin, tissue plasminogen activator, plasminogen, plasmin, urokinase, streptokinase, heparin, thrombomodulin, and Protein C activating agents. An exemplary, and in no way wholly inclusive, listing is provided in Table I below:

TABLE I

| | Selected Bloactive Nucleic Acid Molecules | |
|---|---|---|
| Gene | Clone Type* | Reference and/or GenBank No. |
| Activin | h-cDNA | Mason, Mol. Endo. 3(9):1352-1358, 1989 |
| | | M31668 |
| Adenosine deaminase | h-cDNA | Wiginton, Biochem. 25:8234, 1986 |
| | | M13792 |
| Angiotensinogen I | r-cDNA | Ohkubo, Biochem. 23(16):3603, 1984 |
| | r-gDNA | Tanaka, J Biol. Chem. 259:8063, 1984 |
| | | K02215, L00090, J00704 |
| Antithrombin III | H-cDNA | Bock, Nuc. Acid Res. 10:8113, 1982 |
| | h-cDNA and gDNA | Prochownik, J. Biol. Chem. 258:8389, |
| | | 1983 |
| | | X68793, S52236 |
| Antitrypsin, alpha I | h-cDNA | Kurachi, PNAS 78:6826, 1981 |
| | h-gDNA | Leicht, Nat 297:655, 1982 |
| | RFLP | Cox, AJHG 36:1345, 1984 |
| | | X01683, K01396 |
| Apolipoprotein A-I | h-cDNA, h-gDNA | Shoulders, Nuc. Acid Res. 10:4873, 1982 |
| | RFLP | Karathanasis, Nat. 301:718, 1983 |
| | h-gDNA | Kranthanasis, PNAS 80:6147, 1983 |
| | | J00098, J00099, J00100, J00101, J03222 |
| Apolipoprotein A-II | h-cDNA | Sharpe, Nuc. Acid Res. 12:3917, 1984 |
| | Chr | Sakaguchi, AJHB 36:207S, 1984 |
| | h-cDNA | Knott, BBRC 120:734, 1984 |
| | | X04898 |
| Apolipoprotein C-I | h-cDNA | Knott, Nuc. Acid Res. 12:3909,1984 |
| Apolipoprotein C-II | h-cDNA | Jackson, PNAS 81:2945, 1984 |
| | h-cDNA | Mykelbost, JBC 249:4401, 1984 |
| | h-cDNA | Fojo, PNAS 81:6354, 1984 |
| | RFLP | Humphries, C Gen 26:3 89, 1984 |
| | | J02698 |
| Apolipoprotein C-III | h-cDNA and gDNA | Karathanasis, Nat. 304:371, 1983 |
| | h-cDNA | Sharpe, Nuc. Acid Res. 12:39 17, 1984 |
| | | M28614 |
| Apolipoprotein E | h-cDNA | Brewslow, J Biol. Chem. 257:14639, |
| | | 1982 |
| | | X00199 |
| atrial natriuretic factor | h-cDNA | Oikawa, Nat. 309:724, 1984 |
| | h-cDNA | Nakayama, Nat. 3 10:699, 1984 |
| | h-cDNA | Zivin, PNAS 81 :6325, 1984 |
| | h-gDNA | Seidman, Sci. 226:1206, 1984 |
| | h-gDNA | Nemer, Nat. 312:654, 1984 |
| | h-gDNA | Greenberg, Nat. 312:665, 1984 |
| | | K02043 |
| Chorionic | h-cDNA | Fiddes, Nat. 281:351, 1981 |
| gonadotropin | | V00518 |
| Chorionic | RFLP | Boethby, J. Biol. Chem. 256:5121, 1981 |
| gonadotropin, | | |
| alpha chain | | |
| Chorionic | h-cDNA | Fiddes, Nat. 286:684, 1980 |
| gonadotropin | | |
| Chorionic | h-gDNA | Boorstein, Nat. 300:419, 1982 |
| gonadotropin, | h-gDNA | Talmadge, Nat. 307:37, 1984 |
| beta chain | | M54963, J00117, M38559 |
| Chymosin, pro | bovine-cDNA | Harris, Nuc. Acid Res 10:2177, 1982 |
| (rennin) | | J00003 |
| Complement, factor B | h-cDNA | Woods, PNAS 79:5661, 1982 |
| | h-cDNA and gDNA | Duncan, PNAS 80:4464,1983 |
| Complement C2 | h-cDNA | Bentley DR, PNAS, 81:1212, 1984 |
| | h-gDNA (C2, C4, and | Carroll MC, Nat, 307:237, 1984 |
| | B) | X72875 |
| Complement C3 | m-cDNA | Domdey, PNAS 79:7619, 1983 |
| | h-gDNA | Whitehead, PNAS 79:5021, 1982 |
| | | K02782 |
| Complement C4 | h-cDNA and gDNA | Carroll, PNAS 80:264, 1983 |
| | h-cDNA | Whitehead, PNAS 80:5387, 1983 |
| | | K02404 |
| Complement C9 | h-cDNA | DiSciplo, PNAS 81:7298, 1984 |
| | | K02766 |
| Corticotropin releasing | sheep-cDNA | Furutani, Nat 301:537, 1983 |
| factor | h-gDNA | Shibahara, EMBO J 2:775, 1983 |
| | | J00803, V00571 |
| Epidermal growth | m-cDNA | Gray, Nat 303:722, 1983 |
| factor | m-cDNA | Scott, Sci 21:236, 1983 |
| | h-gDNA | Brissenden, Nat 310:781, 1984 |
| | | Lan, Sci 224:843, 1984 |
| | | J00380 |
| Epoxide dehydratase | r-cDNA | Gonzalez, J Biol. Chem. 256:4697, 1981 |
| Erythropoietin | h-cDNA | Lee-Huang, PNAS 81:2708, 1984 |
| | | S62834 |
| Esterase inhibitor, C1 | h-cDNA | Stanley, EMBO J 3:1429, 1984 |
| Factor VIII | h-cDNA and gDNA | Gitschier, Nat. 312:326, 1984 |
| | h-cDNA | Toole, Nat. 312:342, 1984 |
| | | X01179 |
| Factor IX, Christmas | h-cDNA | Kutachi, PNAS 79:6461, 1982 |
| factor | h-cDNA | Choo, Nat. 299:178, 1982 |
| | RFLP | Camerino, PNAS 81:498, 1984 |
| | h-gDNA | Anson, EMBO J3:1053, 1984 |
| | | J00136 |
| Factor X | h-cDNA | Leytus, PNAS 81:3699, 1984 |
| fibrinogen A alpha | h-cDNA | Kant, PNAS 80:3953, 1983 |
| | | J00128 |
| fibrinogen B beta, | h-gDNA (gamma) | Fornace, Sci. 224:161, 1984 |
| gamma | h-cDNA (alpha | Imam, Nuc. Acid Res. 11:7427, 1983 |
| | gamma) | Fornace, J. Biol. Chem. 259:12826, 1984 |
| | h-gDNA (gamma) | K02569 |
| Factor VII | h-gDNA | J02933, M13232 |
| | h-cDNA | |
| follicle Stimulating | h-cDNA | M16646, M16647 |
| hormone, beta | | |
| follitropin | h-cDNA | M24538, M24539, M24540, M24541 |
| Factor XIII | h-cDNA | X51823 |
| Factor V | h-cDNA | M16967 |
| gastrin releasing | h-cDNA | Spindel, PNAS 81:5699, 1984 |
| peptide | | K02054 |
| glucagon, prepro hamster | c-DNA | Bell, Nat. 302:716, 1983 |
| | h-gDNA | Bell, Nat. 304:368, 1983 |
| | | J00059, V01515 |
| growth hormone | h-cDNA | Martial, Sci. 205:602, 1979 |
| | h-gDNA | DeNoto, Nuc. Acid Res. 9:3719, 1981 |
| | GH-like gene | Owerbach, Sci. 209:289, 1980 |
| | | J00148, K00612 |
| growth hormone, RF | h-cDNA | Gubler, ENAS 80:3411, 1983 |
| (Somatocrinin) | | Mayo, Nat. 306:86:1983 |
| | | X00094 |
| Hemopexin | h-cDNA | Stanley, EMBO J. 3:1429, 1984 |
| | | X02537 |
| Inhibin | porcine-cDNA | Mason, Nat. 318:659, 1985 |
| | | X03265, X03266 |
| insulin, prepro | h-gDNA | Ullrich, Sci. 209:612, 1980 |
| | | V00565 |
| insulin-like growth | h-cDNA | Jansen, Nat. 306:609, 1983 |
| factor I | h-cDNA | Bell, Nat. 310:775, 1984 |
| | | Brissenden, Nat. 310:781, 1984 |
| | | X00173 |
| insulin-like growth | h-cDNA | Bell, Nat. 310:775, 1984 |
| factor II | h-gDNA | Dull, Nat. 310:777, 1984 |
| | | Brissenden, Nat. 310:781, 1984 |
| | | X00910, M17862 |
| interferon, alpha | h-cDNA | Maeda, PNAS 77:7010, 1980 |
| | | V00544 |
| interferon, alpha | h-cDNA (8 distinct) | Goeddel, Nat. 290:20, 1981 |
| (leukocyte), multiple | h-gDNA | Lawn, PNAS 78:5435, 1981 |
| | h-gDNA | Todokoro, EMBO J. 3:1809, 1984 |
| | h-gDNA | Torczynski, PNAS 81:6451, 1984 |
| interferon, beta | h-cDNA | Taniguchi, Gene 10:11, 1980 |
| (fibroblast) | h-gDNA | Lawn, Nuc. Acid Res. 9:1045, 1981 |
| | h-gDNA (related) | Sehgal, PNAS 80:3632, 1983 |
| | h-gDNA (related) | Sagar, Sci. 223:1312, 1984 |
| | | V00546, J00218 |
| interferon, gamma | h-cDNA | Gray, Nat. 295:503, 1982 |
| | h-gDNA | Gray, Nat. 298:859, 1982 |
| | | X13274 |
| interleukin-1 | m-cDNA | Lomedico, Nat. 312:458, 1984 |
| | | X01450 |
| interleukin-2, T-cell | h-cDNA | Devos, Nuc. Acid Res. 11:4307, 1983 |
| growth factor | h-gDNA | Taniguchi, Nat. 302:3 05, 1983 |
| | | Holibrook, PNAS 81:1634, 1984 |
| | | Siegel, Sci. 223:175, 1984 |
| | | X01586 |
| interleukin-3 | m-cDNA | Fung, Nat. 307:233, 1984 |
| | | K01850 |
| kininogen, two forms | bovine-cDNA | Nawa, PNAS 80:90, 1983 |
| | bovine, -cDNA and | Kitamura, Nat. 305:545, 1983 |
| | gDNA | J00010, J00011 |
| luteinizing hormone, | h-gDNA and Chr | Talmadge, Nat. 207:37, 1984 |
| beta subunit | | X00264 |
| luteinizing hormone | h-cDNA and gDNA | Seeburg, Nat. 311:666, 1984 |
| releasing hormone | X01059 | |
| Lymphotoxin | h-cDNA and gDNA | Gray, Nat. 312:721, 1984 |
| | | X01393 |
| mast cell growth factor | m-cDNA | Yokota, PNAS 81:1070, 1984 |
| | | K01668 |
| nerve growth factor, | m-cDNA | Scoff, Nat. 302:538, 1983 |
| beta subunit | h-gDNA | Ullrich, Nat. 303:82 1, 1983 |
| | h-cDNA | Franke, Sci. 222:1248, 1983 |
| | Chr | X52599 |
| oncogene, c-sis, PDGF | h-gDNA | Dalla-Favera, Nat. 295:3 1, 1981 |
| | h-cDNA | X02744 |
| PDGF, chain A | h-cDNA | Clarke, Nat. 208:464, 1984 |
| | | X03795 |
| pancreatic polypeptide | h-cDNA | Boel, EMRO J. 3:909, 1984 |
| and icosapeptide | X00491 | |
| parathyroid hormone | h-cDNA | Hendy, PNAS 78:7365, 1981 |
| | | V00597 |
| Prepro parathyroid | h-gDNA | Vasicek, PNAS 80:2127, 1983 |
| hormone | | |
| Plasminogen | h-cDNA and gDNA | Malinowski, Fed P. 42:1761, 1983 |
| | | X05199 |
| plasminogen activator | h-cDNA | Edlund, PNAS 80:349, 1983 |
| | h-cDNA | Pennica, Nat. 301:214, 1983 |
| | h-gDNA | Ny, PNAS 81:5355, 1984 |
| | | X13097 |
| Prolactin | h-cDNA | Cooke, J. Biol. Chem. 256:4007, 1981 |
| | r-gDNA | Cooke, Nat. 297:603, 1982 |
| | | J00299 |
| Proopiomelanocortin | h-cDNA | DeBold, Sci. 220:721, 1983 |
| | h-gDNA | Cochet, Nat. 297:335, 1982 |
| | | J00292, J00291, J00292 |
| protein C | h-cDNA | Foster, PNAS 81:4766, 1984 |
| | | K02059 |
| Prothrombin | bovine-eDNA | MacGillivray, PNAS 77:5153, 1980 |
| | h-cDNA | Deg. Biochem. 22(9):2087, 1983 |
| | | J00041, V00595 |
| relaxin | h-gDNA | Hudson, Nat. 301:628, 1983 |
| | h-cDNA (2 genes) | Hudson, EMBO J. 3:2333, 1984 |
| | Chr | Crawford, EMBO J. 3:2341, 1984 |
| | | X00948, X00949 |
| renin, prepro | h-cDNA | Imai, PNAS 80:7405, 1983 |
| | h-gDNA | Hobart, PNAS 81:5026, 1984 |
| | h-gDNA | Miyazaki, PNAS 81:5999, 1984 |
| | Chr | Chirgwin, SCMG 10:415, 1984 |
| | | L00064-L00073 |
| Somatostatin | h-cDNA | Shen, PNAS 79:4575, 1982 |
| | h-gDNA | Naylot, PNAS 80:2686, 1983 |
| | | J00306 |
| tachykinin, prepro | bovine-cDNA | Nawa, Nat. 306:32, 1983 |
| | h-cDNA | X00075, X54469 |
| substances P & K | bovine-cDNA | Nawa, Nat. 312:72 9, 1984 |
| | h-cDNA | X00075, X54469 |
| Streptokinase | cDNA | E01413 |
| Thrombomodulin | h-cDNA | D00210 |
| Thrombopoietin | h-cDNA | L36052 |
| Urokinase | h-cDNA | Verde, PNAS 81:4727, 1984 |
| | | D00244 |
| vasoactive intestinal | h-cDNA | Itoh, Nat. 304:547, 1983 |
| peptide, prepro | | DeLamarter, Peptides 6(supp. 1):95, 1985 |
| | | M36634 |
| Vasopressin | h-cDNA | Rehbein, Biol. Chem. 367(8):695, 1986 |

Therapeutic nucleic acids and polypeptides used in the invention include recombinantly produced fusion proteins. Fusion proteins may consist of two or more polypeptides or fragments thereof. In certain embodiments, fusion proteins comprise a therapeutic polypeptide tagged with an immunogenic epitope such as the FLAG epitope (Kodak) which can be used to examine expression and delivery of the therapeutic protein by immunological methods known in the art such as ELISA, western blot or radioimmunoassay (RIA). In specific embodiments, fusion proteins contain a targeting moiety introduced to promote efficient uptake of a fused therapeutic polypeptide into target cells. Examples of targeting moieties include immunoglobulins and ligands which bind target cell surface receptors (e.g., polypeptides reactive with the FGF receptor, U.S. Ser. No. 08/718,904).

In addition, the bioactive nucleic acid delivered may express RNA molecules such as antisense or ribozymes or polypeptides such as antibodies, dominant negative proteins or protein variants, as described earlier. The bioactive nucleic acid or polypeptide may also correspond to soluble receptors of proinflammatory cytokines.

It is within the scope of the invention to provide one, two or more nucleic acids and/or polypeptides. Therapeutic regimens may include either a single treatment with one or multiple nucleic acids and/or polypeptides, two or more treatments with one or multiple nucleic acids or polypeptides, or multiple treatments with one or more nucleic acids or polypeptides. Appropriate therapeutic regimens are designed considering, amongst other factors, the particular disease being treated and its stage or severity, the physical characteristics of the patient, the particular nucleic acids or polypeptides being supplied, the type of bioreactor matrix employed, and the mode of nucleic acid delivery.

Nucleic acids encoding products of interest may be obtained by a variety of molecular biology techniques well known to those skilled in the art. For example, cDNA or genomic libraries may be screened using probes based on published nucleic acid sequences or antibodies directed against polypeptide products of interest. Polymerase chain reaction (PCR) techniques may also be employed to generate DNA fragments of interest from DNA libraries, genomic DNA or RNA samples, for example. Alternatively, DNA fragments may be obtained commercially. Nucleic acid sequences of interest are available in the art and from the GenBank databases.

Nucleic acids may be propogated and produced in a substantially pure form suitable for therapeutic use by any means available in the art. Nucleic acids may be cloned into a variety of vector systems that provide for replication and production of large amounts of DNA in host cells, including plasmids, viruses, episomes, cosmids and bacteriophage. Vectors are transferred to host cells by known methods including transfection, transformation or infection. Suitable host cells may include bacteria such as E. coli, yeast, plant cells, mammalian tissue culture cells or baculovirus. Techniques for propogating and purifying recombinant nucleic acid vectors are well known in the art and are described in Sambrook et al. 1992 Molecular Cloning, A Laboratory Manual, Cold Spring Harbor Laboratory, NY and Ausubel et al., 1989 Current Protocols in Molecular Biology, Greene Publishing Associates and Wiley Interscience, NY. Alternatively, nucleic acid molecules may be chemically synthesized.

Nucleic acid vectors may contain elements necessary for directing the transcription and/or translation of genes of interest within cells associated with the bioreactor. Thus, the nucleic acids encoding products of interest may be operatively linked with a variety of promoter/enhancer and translational elements which direct expression. Expression elements may differ in their tissue specificities and their relative strengths. Depending on the system utilized, any one of a number of suitable transcription and translation elements may be used. The promoter may be in the form of a promoter which is naturally associated with the gene of interest. Alternatively, the nucleic acid may be under control of a heterologous promoter not normally associated with the gene. For example, tissue specific promoter/enhancer elements may be used to direct expression of the transferred nucleic acid in repair cells. In certain instances, the promoter elements may drive constitutive, event-specific (see for example U.S. Pat. Nos. 5,681,746 and 5,716,826, incorporated herein by reference), tissue-specific or inducible expression of the nucleic acid of interest. Mammalian promoters may be used, as well as viral promoters capable of driving expression in mammalian cells.

It is also within the scope of the invention to use multiple genes, combined on a single genetic construct under control of one or more promoters, or prepared as separate constructs of the same or different types. Thus, an almost endless combination of different nucleic acids and genetic constructs may be employed. Certain gene combinations may have synergistic effects on cell growth or therapeutic treatment of a disease or related condition. Any and all such combinations fall within the scope of the invention.

RNAs utilized in the invention may be produced by any means known in the art, such as in vitro synthesis from a vector directing transcription from a promoter such as T3, T7 or Sp6, as described in Ausubel et al., 1989, Current Protocols in Molecular Biology, Greene Publishing Associates and Wiley Interscience, NY.

Nucleic acid molecules encoding growth promoting and bioactive agents may be utilized in gene delivery vehicles. Any method of gene delivery available in the art may be utilized according to the present invention. Gene delivery vehicles may be of viral or non-viral origin (see generally, Jolly, Cancer Gene Therapy 1:51-64, 1994; Kimura, Human Gene Therapy 5:845-852, 1994; Connelly, Human Gene Therapy 1:185-193, 1995; and Kaplitt, Nature Genetics 6:148-153, 1994). Expression of coding sequences can be controlled using endogenous mammalian or heterologous promoters and may be either constitutive or regulated. Nucleic acids used according to the invention may be stably integrated into the genome of the cell or may be maintained in the cell as separate episomal segments of DNA.

The present invention can employ recombinant retroviruses which are constructed to carry or express a selected nucleic acid molecule of interest. Methods of producing recombinant retroviral virions suitable for gene therapy have been extensively described (see, e.g., Mann et al., Cell 33:153-159, 1983 and Nikolas and Rubenstein, Vectors: A survey of molecular cloning vectors and their uses, Rodriquez and Denhardt (eds.), Stoneham: Butterworth, 494-513, 1988).

The present invention also employs viruses such as alphavirus-based vectors, adenovirus and parvovirus that can function as gene delivery vehicles. In certain embodiments of the invention, adenovirus or adenovirus-derived vectors are utilized for introduction of one or more nucleic acid molecules. Examples of vectors utilized by the invention include intact adenovirus, replication-defective adenovirus vectors requiring a helper plasmid or virus, and adenovirus vectors with their native tropism modified or ablated including adenoviral vectors containing a targeting ligand. In specific embodiments, the targeting ligand is a polypeptide reactive with a cell surface receptor such as an FGF receptor. Vector compositions, systems and methods for using these adenovirus vectors are disclosed in WO 98/40508 which is incorporated by reference in its entirety.

Packaging cell lines suitable for use with the above-described viral and retroviral vector constructs may be readily prepared and used to create producer cell lines (also termed vector cell lines) for the production of recombinant vector particles. Further as those of skill in the art would readily appreciate many viral delivery agents may be utilized for example, and in no way limiting these would include those referenced above as well as herpes virus, lentivirus, and hybrid viral constructs.

Examples of non-viral methods of gene delivery vehicles and methods which may be employed according to the invention include liposomes of several types (see, e.g., Wang et al. PNAS 84: 7851-7855, 1987), polycationic condensed DNA linked or unlinked to adenovirus (see e.g., Curiel, Hum. Gene Ther. 3:147-154, 1992); ligand linked DNA, (see, e.g., Wu, J. Biol. Chem. 264:16985-16987, 1989); deposition of photopolymerized hydrogel materials; hand-held gene transfer particle gun, as described in U.S. Pat. No. 5,149,655; ionizing radiation as described in U.S. Pat. No. 5,206,152 and WO 92/11033; and nucleic charge neutralization or fusion with cell membranes. Additional approaches are described in Philip, Mol. Cell Biol. 14:2411-2418, 1994, and in Woffendin, Proc. Natl. Acad. Sci. 91:1581-1585, 1994. Conjugates comprising a receptor-binding internalized ligand capable of delivering nucleic acids may also be used according to the present invention. Conjugate-based preparations and methods of use thereof are described in WO 96/36362 which is hereby incorporated by reference in its entirety. Other non-viral delivery methods include, but are not limited to, mechanical delivery systems such as the approach described in Woffendin et al., Proc. Natl. Acad. Sci. USA 91(24):11581-11585, 1994 and naked DNA protocols. Exemplary naked DNA introduction methods are described in WO 90/11092 and U.S. Pat. No. 5,580,859.

In other embodiments, methods of the invention utilize bacteriophage delivery systems capable of transfecting eukaryotic cells. Bacteriophage-mediated gene transfer systems are described in WO 99/10014 which is incorporated in its entirety. Phage delivery vehicles may express a targeting ligand on their surface which facilitates receptor-mediated gene delivery.

Therapeutic Uses of Bioreactors

The compositions and methods of the current invention can be used to treat a variety of diseases and related medical conditions amenable to systemic application of therapeutic nucleic acid and polypeptide products. Diseases and related conditions amenable to treatment may be both short term or temporary, or they may be chronic or permanent conditions resulting from disease or injury. Diseases associated with plasma protein deficiencies are particularly amenable to treatment according to the invention. In certain embodiments, compositions and methods of the invention will provide systemic delivery of therapeutic replacement proteins to compensate for a shortage of an endogenous protein. Examples of diseases or related indications which may be treated with therapeutic replacement proteins according to the invention include conditions associated with renal dialysis, hemophilia, hemoglobinopathies, thalassemias, anemia, lipid storage diseases, mucopolysaccharidoses, diabetes, and hypercoagulability. In other embodiments, the compositions and methods of the invention will be used to treat symptoms associated with a disease. For one example, the pain associated with arthritis may be treated using nucleic acids encoding anticoagulants.

Representative examples of candidate disease targets are hemophilia A which results from a deficiency of factor VIII and erythropoietin (EPO)-responsive anemias. These diseases are currently treated by regular injections of purified or recombinant proteins, an expensive and inconvenient therapy. Bioreactors which provide continuous therapeutic levels of factor VIII or EPO will provide significant clinical, economic and lifestyle benefits for patients suffering from these diseases and thus fill a significant need in the medical arts.

The clinical severity of hemophilia A correlates inversely with circulating factor VIII levels. To achieve therapeutic benefit, factor VIII serum levels of less than 10-20 ng/ml are required. Factor VIII levels as little as 5% of normal will convert a patient with severe hemophilia A and frequent spontaneous bleeding to a person with mild disease, bleeding only with major trauma. The cDNA for human Factor VIII has been cloned, and the large internal B domain has been shown to be dispensable for clotting activity. Therefore, wild type Factor VIII or B domain deleted Factor VIII may be used as a potential gene therapeutic. Other potential therapeutic molecules involved in the clotting cascase include: Factor V (FV), Factor VII (FVII), Factor IX (FIX), Factor X, (FX), Factor XI (FXI), Factor XIII (FXIII), thrombin, fibrin, fibrin stabilizing factor, and von Willebrand Factor.

Certain anemias such as those associated with chronic renal failure, HIV infection, or cancer chemotherapy exhibit dramatic reductions in the production of EPO, a glycoprotein hormone produced by the kidney which stimulates bone marrow to produce red blood cells. Current therapy is designed to maintain the hematocrit at levels sufficient to permit a more active lifestyle but generally fall short of utilizing optimal amounts of recombinant EPO because of its high cost. In addition, high dose recombinant EPO is being investigated for the treatment of some hemoglobinopathies. The use of an EPO-producing bioreactor would provide continuous production of optimal therapeutic levels of EPO, while also permitting its removal if exogenous EPO replacement was no longer needed, or if EPO levels exceeded those needed to maintain a normal hematocrit level.

Patients suffering from diseases or disorders associated with hypercoagulability or at risk for stroke may be treated with anticoagulants such as thrombomodulin, Protein C activating agents, Protein C, and antithrombin or fibrinolytic agent such as tissue plasminogen activator, plasminogen, plasmin, urokinase, and streptokinase, for example.

Pro-inflammatory, infectious, and autoimmune diseases and related conditions, as well as immune responses to disease or injury may be treated according to the present invention. Candidate diseases for treatment include, for example, multiple sclerosis, arthritis, hepatitis including hepatitis B and hepatitis C, inflammatory bowel diseases, cyclic neutropenia, and thrombocytopenia. Therapeutic nucleic acids and/or polypeptides which may be used to treat these diseases include, for example, beta or gamma-interferon for multiple sclerosis, beta or gamma-interferon for hepatitis, antibodies to proinflammatory cytokines such as interleukin-l and tumor necrosis factors, hematopoietic growth stimulants, granulocyte-colony stimulating factor (G-CSF) for cyclic neutropenia, thrombopoietin (TPO), megakaryocyte growth and development factor (MGDF) or interleukin-12 for thrombocytopenia or any therapeutic antibody for systemic use.

In certain embodiments, the invention may be used to treat cancer and other proliferative disorders or related conditions. Tumoricidal antibodies or ligands which interact with tumor specific antigens or cell surface receptors may be delivered systemically by the present invention. Systemic immunotherapy according to the invention is advantageous since it allows treatment of metastatic tumor cells which may not be clinically detectable. Treatment according to the invention may also be desirable in addition to traditional cancer treatments such chemotherapy, radiation therapy or bone marrow transplantation. In these instances, treatment with a bioactive nucleic acid or polypeptide encoding a cytokine or growth factor such as G-CSF will promote repopulation of depleted hematopoietic cells.

Additional examples of therapeutic polypeptides and diseases which may be treated according to the present invention include using insulin growth factor-i to treat amyotrophic lateral sclerosis (ALS), granulocyte colony stimulating factor to treat neutropenia, alpha-1 proteinase inhibitor to treat alpha-1 antitrypsin deficiency, calcitonin to treat osteoporosis, carbonic anhydrase to treat osteopetrosis, purine nucleoside phosphorylase (PNP) and adenosine deaminase (ADA) to treat immune deficiency diseases diseases, globins to treat sickle cell anemia, and nerve growth factor to treat peripheral neuropathy. Certain diseases such as Crohns's IBD, ulcerative colitis, rheumatoid arthritis, and cancer may prove particularly amenable to treatment with antibodies, particularly monoclonals and humanized antibodies (e.g., anti-Her2 antibodies).

The invention may also be used to treat infertility and for birth control. Patients may be treated according to the invention with peptide hormones that affect fertility. For example, patients may be treated systemically with luteinizing hormone, follicle-stimulating hormone and/or releasing hormone.

In other embodiments, the invention may be used to provide a structural support for tissue or organ reconstruction or enhancement. Bioactive nucleic acid molecules with anti-inflammatory activities may be provided systemically according to the invention to curtail undesirable immune responses to the implant.

Kits

Materials and reagents utilized in various aspects of the present invention may be assembled in a kit. Kits for the production of a BI-GENE™ device and kits for the production of a BI-GENE™ coated device are provided by the present invention. A kit for the production of a BI-GENE™ or DUO-GENE™ device may comprise an appropriate container, a biocompatible matrix, a growth stimulating agent and a bioactive agent. A kit for the production of a BI-GENE™ coated device may comprise a device coated with a biocompatible matrix, a growth stimulating agent and a bioactive agent. In preferred embodiments, the growth promoting agent and the bioactive agent are nucleic acid molecules. In certain preferred embodiments, the kits include nucleic acid molecules which encode growth stimulating agents and bioactive agents. In some instances, a kit may comprise of more than one growth stimulating agent and/or bioactive agent. In other instances, a kit may comprise of a nucleic acid encoding a bioactive agent and a polypeptide growth promoting agent. In yet another embodiment, a kit may comprise of a nucleic acid encoding a bioactive agent, and the user of the kit will supply the growth promoting agent according to the invention. The container may be a suitable box, or it may be an inhalant, syringe, pipette, dropper, or any other such apparatus, from which the formulation may be applied to a wound site, or even applied to or mixed with other components of the kit.

Components of the kit may be provided in a dried or lyophilized form or in one or more liquid solutions. When the components are provided in liquid solution, the liquid solution is preferably a sterile, aqueous solution. When components are provided in a dried form, the dried form may be capable of reconstitution upon addition of a suitable solvent or capable of reconstitution by bodily fluids post-implantation. Dried components may be reconstituted prior to implantation. In certain embodiments, dried solutions may be reconstituted after implantation by administering a suitable solvent, e.g., by syringe.

Kits will generally be packaged in an outer container suitable for commercial sale and distribution. Kits of the invention may also comprise or be packaged with instructions for use and instruments for assisting in use. Examples of instruments include syringes, inhalants, pipettes, vials, forceps, measuring spoons, eye droppers or any other medically approved delivery device. Kits may also comprise reagents to be used for analyzing polypeptide production by the implanted BI-GENE™ device or BI-GENE™ coated device. One example of reagents which may be included for this purpose are antibodies which recognize bioactive molecules expressed by the bioreactor.

### EXAMPLES

### Example 1

### New Tissue Formation In Vivo Within a Biocompatible Substance

PVA sponges were implanted subcutaneously into rats on day 0 and injected on day 4 with collagen containing 10⁹ pfu adenovirus encoding luciferase (AdLuc) (Rogers et al., Tumor Targeting 3:25-31, 1998) or PDGF-BB (AdPDGF) (Liechty et al., J. Invest. Dermatol. 113(3):375-383, 1999). At day 10 post-implantation, sponges were removed and processed. Expression of PDGF-BB was confirmed by ELISA.

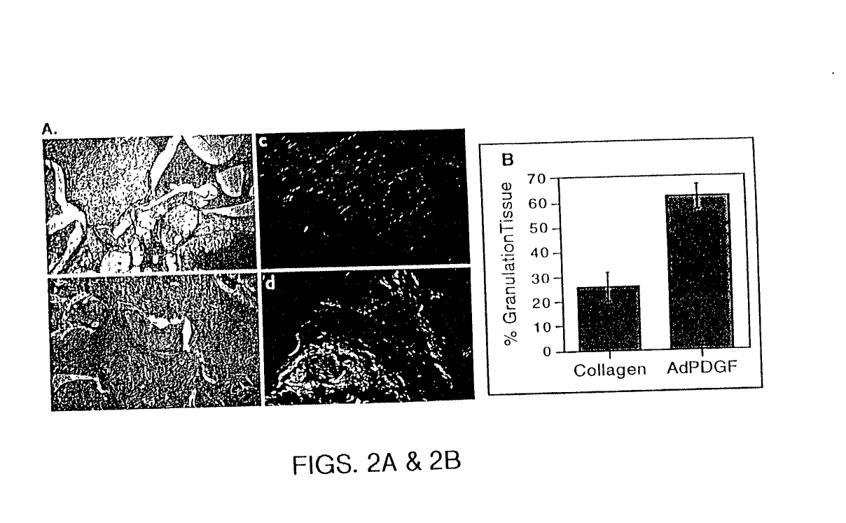

Sections were stained with Alcian blue to detect infiltrating cells and glycosaminoglycans or Sirius red to detect mature collagen bundles. As demonstrated in FIG. 2A, increased cellularity and vascularity present were observed in AdPDGF sponges.

Formalin-fixed, paraffin-embedded sponge sections were stained using Masson's Trichrome. Image-Pro Plus software (Media Cybernetics, Silver Spring, Md.) was used to differentiate new tissue from sponge matrix based on pixel density. As depicted in FIG. 2B, percent new tissue area/total area was calculated as approximately 25% for collagen containing AdLuc and 60% for collagen containing AdPDGF.

### Example 2

### Secondary Transduction of AdPDGF Transduced Fibroblasts

The ability of AdPDGF transduced human dermal fibroblasts to be subsequently transduced with an adenovirus vector encoding luciferase (AdLuc) was examined.

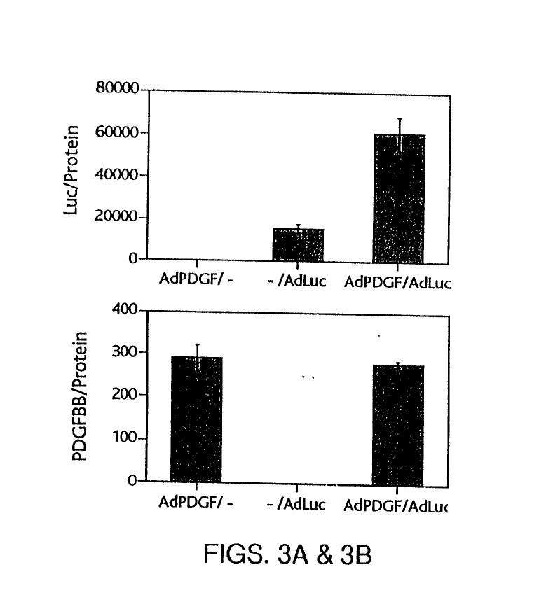

WS-1 human dermal fibroblasts were infected with AdPDGF-B (MOI 500). Control wells received no treatment. Forty-eight hours later, AdLuc was added to the indicated wells at MOI 100. After an additional 24 hours, culture supernatants were harvested for PDGF-BB ELISA, and cell extracts were prepared for evaluation of luciferase activity. Expression of each transgene was normalized to total cellular protein in each well as determined by BCA assay. See FIGS. 3A and 3B.

The data demonstrate that 1) levels of PDGF-BB expression were unaffected by subsequent transduction with AdLuc, and 2) cells “primed” by transduction with AdPDGF expressed significantly higher levels of luciferase than cells that were not treated with AdPDGF-B. The results demonstrated that populations of PDGF-BB-stimulated fibroblasts are readily transduced by a second Ad vector and can express the second transgene at high levels.

### Example 3

### Animal Models of In Situ Bioreactor

Animal models of in situ bioreactors are established using specific pathogen-free male CD rats from 400-450 gram body weight (outbred strain, Charles River Laboratories, Wilmington, Mass.) and specific pathogen-free female C57B1/6 mice (Charles River Laboratories).

An E1-, E3-deleted human adenovirus type 5 vector encoding human PDGF-BB (AdPDGF) under control of the CMV is utilized (Leichty et al., (supra)). Virus is prepared as chromatographically purified stocks of defined plaque forming units (PFU) and viral particle concentration standard methodologies. Collagen gels containing vectors are prepared on ice by the sequential addition of purified bovine dermal type I collagen (Cell Prime, Collagen Corp., Freemont, Calif.), water, 10× Minimal Essential Medium (GIBCO/BRL, Grand Island, N.Y.), 7.5% NaHCO₃, FBS, and vector. The final formulation is 1.45 mg/ml collagen, 2.2 mg/ml NaHCO₃, 10% FBS.

Characterization of the Persistence of PDGF-BB-augmented Neo-tissue Organoids.

Male Sprague-Dawley rats (400-450 g) are anesthetized with 60 mg/kg ketamine and 8 mg/kg xylazine i.p., and the ventral surface shaved and prepared using betadine, followed by 70% isopropyl alcohol. Under sterile conditions, 3 bilateral sets of longitudinal, full thickness incisions are made through the skin and panniculus carnosus. The incisions are 5 mm in length, placed 20 mm from the midline on both sides, and are 10 mm apart longitudinally. This allows for the reproducible placement of 6 PVA sponges (1.2 cm diameter, 3 mm thick, grade 3 PVA sponge from M-PACT, Eudora, KS) per rat. Preliminary studies using tracer-loaded sponges have established that no cross-diffusional effects between sponges are seen with these placement conditions (data not shown). Incisions are closed with wound clips. On day 4 post-implantation, 200 μl of AdPDGF (1×10⁹ pfu), or saline as a control, are injected into the center of each sponge. At various experimental time points after AdPDGF injection, selected rats are euthanized using 60 mg/kg pentobarbital intraperitoneally, and sponges dissected free from the underlying and overlying musculature and adventitia while leaving the capsule intact. Upon removal, sponges are weighed to get a general indication of new tissue formation. Paraffin sections from the center of each sponge are stained using Masson's Trichrome and evaluated for semi-quantitation of new tissue, vascularity, and ECM deposition.

Optimization of gene levels can be tested by a variety of routine methods, for example, the persistence of the de novo tissue induced by AdPDGF administration 4 days post-sponge implantation may be determined. In this regard, two treatment groups are utilized: 1) Collagen; 2) AdPDGF in collagen (control of AdLuc in collagen). Sponges are harvested for histologic examination at 10, 14, 21, 28, and 42 days post implantation. Sponges are also processed for evaluation of PDGF-BB expression by ELISA. Specifically, 96-well plates (Costar, Cambridge, Mass.) are coated for 12 h with rhPDGF Rβ/Fc chimera (R&D Systems, Minneapolis, Minn.) at 50 pg/50 μl PBS/well. Wells are then rinsed with TBS containing 0.05% Tween 20 (TBST) and blocked in TBST containing 2% BSA. Following incubation with experimental samples or a standard curve constructed from rhPDGF-BB (R&D Systems), wells are rinsed with TBST, and bound human PDGF-BB detected using anti-PDGF-BB (R&D Systems). Bound antibody is detected using AP-anti-rabbit IgG (Boehringer Mannheim, Indianapolis, Ind.), and conversion of pNPP substrate is determined at a detection wavelength of 405 nm and a reference wavelength of 490 nm. PDGF-BB levels are normalized to total cellular protein as determined by BCA assay (Pierce).

In a second experiment, the ability of a second dose of AdPDGF to replenish/sustain the vascularized organoid is determined. At the time point (day Y) at which there is evidence for tissue involution following the initial dose of AdPDGF, a second dose is administered and the effects are examined histologically 7, 14, and 21 days after administration of the second dose. Treatment groups include: 1) Collagen (day 4); 2) AdPDGF (day 4); 3) AdPDGF (day 4) +AdPDGF (day Y).

Analysis of the Ability of PDGF-BB-augmented Organoids to Support Expression of a Second Gene.

Previous studies have demonstrated a high cellularity and concomitant matrix deposition by AdPDGF-treated sponges 10 days post-implantation (day 6 after AdPDGF dosing). Sponges treated on day 4 with AdPDGF are subsequently treated with a range of doses of AdLuc on days 4, 7, 10, or 14 post-implantation. Luciferase activities are measured at 48 h post AdLuc treatment. Treatment groups are: 1) Collagen (day 4)+AdLuc (day 4, 7, 10, or 14); 2) AdPDGF (day 4)+adenovirus vehicle (day 4, 7, 10, or 14); 3) AdPDGF (day 4)+AdLuc (day 4, 7, 10, or 14). Sponges are processed by mechanical homogenization in 0.2% Triton X-100, 100 mM potassium phosphate, pH 7.8 using the FastPrep tissue processor (Bio101, Vista, Calif.). Homogenized samples are subjected to 1 freeze/thaw cycle and then clarified by centrifugation at 12-14,000×g, 4° C., 20 min. A two-fold dilution series of the clarified supernatant is assayed using Promega's luciferase assay kit and luciferase levels are normalized to total cellular protein. Sample activities are extrapolated by linear regression from a standard curve prepared with purified recombinant luciferase.

In a second in vivo experiment, persistence of luciferase gene expression is determined. Luciferase is inherently unstable, therefore detectable activity reflects recent synthesis. AdLuc is injected into PVA sponges at the time point (day X) that gives the highest level of luciferase expression as determined in the preceding experiment. Sponges are harvested at 2, 4, 7 and 10 days after administration of AdLuc and processed for luciferase activity measurements as described above. Treatment groups are: 1) Collagen (day 4)+AdLuc (day X); 2) AdPDGF (day 4)+adenovirus vehicle (day X), 3) AdPDGF (day 4)+AdLuc (day X). Five additional time points are also evaluated.

The ability of this replenished tissue to support luciferase gene expression is determined. The timing of the second AdPDGF dosing is based on the results obtained in prior experiments. In the initial experiment, only one dose of AdLuc is administered after the second AdPDGF dose using the dosing schedule that gives the highest luciferase expression as defined in the preceding experiments. The levels of luciferase activity supported by the replenished tissue are compared to that supported by the neo-tissue. Luciferase activities are measured 48 hours after AdLuc administration. Treatment groups are: 1) AdPDGF (day 4)+AdPDGF (day Y); 2) AdPDGF (day 4)+AdPDGF (day Y)+AdLuc (day Z); 3) AdPDGF (day 4)+AdLuc (day Z).

The PVA sponge model is also evaluated in C57B1/6 mice. Two sponges are implanted per mouse. Sponges treated on day 4 with AdPDGF are subsequently treated with AdLuc on days 4, 7, 10, or 14 post-implantation. Luciferase activities are measured at 48 h post AdLuc treatment. Treatment groups are: 1) collagen (day 4)+AdLuc (day 4, 7, 10, or 14); 2) AdPDGF (day 4)+adenovirus vehicle (day 4, 7, 10, or 14); 3) AdPDGF (day 4)+AdLuc (day 4, 7, 10, or 14).

Evaluation of Transgene-encoded Systemic hFVIII Levels and Hematocrit Changes Induced by FVIII and EPO DNA Vectors, Respectively.

The ability of the organoids to express secreted, therapeutic proteins is assessed. An adenoviral vector encoding B domain-deleted (BDD) human FVIII is generated, according to routine methods. Both adenoviral and nonviral vectors encoding human and mouse EPO have been described by (Tripathy et al., Proc. Natl. Acad. Sci. (USA) 93:10876-10880, 1996; Svensson et al., Hum. Gen. Ther. 8:1797-1806, 1997).

C57B1/6 mice are employed instead of rats since it is easier to detect therapeutic levels of FVIII or EPO-induced changes in HCTs, and since this strain has been widely used in gene therapy studies. In addition, published work evaluating other FVIII and EPO gene therapy strategies have employed mouse models. PVA sponges are implanted s.c. into mice on day 0, treated on day 4 with AdPDGF, and then treated with FVIII or EPO vectors at the time point (day X) resulting in the highest levels of luciferase expression. Optimization of the timing for administration of the serum protein transgene and for maximizing serum levels of the transgene product is determined experimentally. Four sponges are implanted per mouse (2 ventrally and 2 dorsally) and all sponges in a single animal receive the same treatment in order to maximize the potential to detect plasma levels of FVIII or EPO-enhanced HCTs. Blood collection for interim analyses is performed by orbital bleed on days 4 and 7 for FVIII or days 10 and 14 for EPO. For EPO experiments, to confirm that increases in HCT are due to increases in RBC, hemoglobin levels and reticulocyte percentages are selectively determined in some mice. At sacrifice, blood is collected by cardiac puncture and animals euthanized by exsanguination. Further, it may be beneficial to monitor for antibodies in any experiments that extend beyond 10 days, and to employ mouse transgenes when necessary. Plasma levels of FVIII are determined by hFVIII-specific ELISA according to the protocol provided by the manufacturer (FVIII Coatest Kit; Source Chromogenix; or ELISA-Immunobind Factor VIII ELISA; American Diagnostica). For measurement of the therapeutic effect of EPO, blood is collected into heparinized tubes and the hematocrit determined.

From the foregoing it will be appreciated that, although specific embodiments of the invention have been described herein for purposes of illustration, various modifications may be made without deviating from the spirit and scope of the invention. Accordingly, the invention is not limited except as by the appended claims. In addition, all patents, patent applications, and other references referred to herein are incorporated by reference in their entirety.

1. An in situ bioreactor adapted for systemic delivery of bioactive agents, comprising a first nucleic acid molecule encoding a cell growth stimulating agent, a second nucleic acid molecule encoding a bioactive agent, and a biocompatible substance capable of cellular infiltration.

2. The bioreactor of claim 1, wherein the cell growth stimulating agent is selected from the group consisting of: a transcription factor, a chemotactic factor, an angiogenic factor, an antisense molecule, a ribozyme, an anti-apoptotic molecule, a growth factor, a cytokine, an extracellular matrix moleule, a cell adhesion protein, a cell retention agent, and a cell surface receptor.

3. The bioreactor of claim 2, wherein the first nucleic acid molecule encodes a growth factor or cytokine.

4. The bioreactor of claim 3, wherein the growth factor is selected from the group consisting of: transforming growth factor (TGF), fibroblast growth factor (FGF), platelet derived growth factor (PDGF), insulin like growth factor (IGF), vascular endothelial growth factor (VEGF), hepatocyte growth factor (HGF), epidermal growth factor (EGF), colony stimulating factor (CSF), angiopoietin, interleukin and bone morphogenic factor (BMP) family members.

5. The bioreactor of claim 4, wherein the growth factor comprises one or more PDGF family members.

6. The bioreactor of claim 5, wherein the growth factor is PDGF-B.

7. The bioreactor of claim 4, wherein the growth factor is HGF.

8. The bioreactor of claim 4, wherein the growth factor comprises one or more FGF family members.

9. The bioreactor of claim 8, wherein the growth factor is FGF-2.

10. The bioreactor of claim 9, wherein the FGF-2 is a mutated FGF-2.

11. The bioreactor of claim 8, wherein the growth factor is FGF6.

12. The bioreactor of claim 4, wherein the growth factor is one or more TGF family members.

13. The bioreactor of claim 12, wherein the growth factor is selected from the group consisting of TGF-β1, TGF-β2, and TGF-β3.

14. The bioreactor of claim 2, wherein the cell growth stimulating agent is an antisense molecule.

15. The bioreactor of claim 2, wherein the cell growth stimulating agent is a ribozyme molecule.

16. The bioreactor of claim 2, wherein the cell growth stimulating agent is an anti-apoptotic agent.

17. The bioreactor of claim 16, wherein the anti-apoptotic agent is Bcl-2.

18. The bioreactor of claim 16, wherein the anti-apoptotic agent is Bcl-xL.

19. The bioreactor of claim 16, wherein the anti-apoptotic agent is A20.

20. The bioreactor of claim 2, wherein the tissue growth stimulating factor is a transcription factor.

21. The bioreactor of claim 20, wherein the transcription factor is an activator or a repressor.

22. The bioreactor of claim 21, wherein the transcription factor is selected from the group consisting of: NF-κB, E2F, DP1, AP-1, AP-2, myc, p53, Sp1, NFAT, CBP, C/EBP, and nuclear hormone receptor family members.

23. The bioreactor of claim 1, wherein the bioreactor further comprises a cell retention agent.

24. The bioreactor of claim 1, wherein the bioreactor further comprises a nucleic acid encoding a cell retention agent.

25. The bioreactor of claims 23 or 24, wherein the cell retention agent is selected from the group consisting of: macrophage migration inhibitory factor (MIF), extracellular matrix molecules, and cell adhesion molecules.

26. The bioreactor of claim 1, wherein the second nucleic acid molecule encodes a hormone.

27. The bioreactor of claim 26, wherein the hormone is selected from the group consisting of: growth hormone, insulin, atrial natriuretic peptide (ANP), luteinizing hormone, follicle-stimulating hormone, releasing hormones, inhibin, relaxin, activin, and follitropin.

28. The bioreactor of claim 27, wherein the hormone is insulin.

29. The bioreactor of claim 1, wherein the second nucleic acid molecule encodes a bioactive agent selected from the group consisting of: Factor V (FV), Factor VII (FVII), Factor VIII (FVIII), Factor IX (FIX), Factor X, (FX), Factor XI (FXI), Factor XIII (FXIII), erythropoietin (EPO), growth hormone (GH), adenosine deaminase, thrombopoietin, purine nucleoside phosphorylase (PNP), Protein C, Protein S, an interleukin, an interferon, a globin, an antibody, and an antibody fragment.

30. The bioreactor of claim 1, wherein the second nucleic acid molecule encodes a fibrinolytic agent.

31. The bioreactor of claim 30, wherein the fibrinolytic agent is selected from the group consisting of: tissue plasminogen activator, plasminogen, plasmin, urokinase, and streptokinase.

32. The bioreactor of claim 1, wherein the second nucleic acid molecule encodes an anticoagulant.

33. The bioreactor of claim 32, wherein the anticoagulant is selected from the group consisting of: thrombomodulin, Protein C activating agents, Protein C, and antithrombin.

34. The bioreactor of claim 1, wherein the second nucleic acid encodes a coagulant.

35. The bioreactor of claim 34, wherein the coagulant is selected from the group consisting of: thrombin, fibrinogen, fibrin stabilizing factor, Factor IX, Factor VIII, von Willebrand factor, and Factor X.

36. The bioreactor of claim 35, wherein the anticoagulant is Factor IX.

37. The bioreactor of claim 29, wherein the second nucleic acid molecule encodes FVIII.

38. The bioreactor of claim 29, wherein the second nucleic acid molecule encodes EPO.

39. The bioreactor of claim 1, wherein the first and second nucleic acid molecules are operably linked to promoters.

40. The bioreactor of claim 39, wherein the promoters may be independently selected from group consisting of constitutive, inducible, event specific, and tissue specific promoters.

41. The bioreactor of claim 1, wherein the nucleic acid molecule is in the form of a plasmid, or a recombinant insert in the genome of a virus.

42. The bioreactor of claim 41, wherein the virus is selected from the group consisting of an adenovirus, an adeno-associated virus, and a retrovirus.

43. The bioreactor of claim 42, wherein the virus is an adenovirus.

44. The bioreactor of claim 1, wherein the nucleic acid molecule is associated with a condensing agent.

45. The bioreactor of claim 44, wherein the condensing agent is a polycationic agent.

46. The bioreactor of claim 1, wherein at least one nucleic acid molecule is associated with a cell surface binding moiety.

47. The bioreactor of claim 46, wherein the binding moiety is a polypeptide reactive with a fibroblast growth factor receptor.

48. The bioreactor of claim 46, wherein the polypeptide reactive with an FGF receptor is selected from the group consisting of FGF-1, FGF-2, FGF-3, FGF-4, FGF-5, FGF-6, FGF-7, FGF-8, FGF-9, FGF-10, FGF-11, FGF-12, FGF-13, FGF-14, FGF-15, FGF-16, FGF-17, FGF-18, FGF-19, FGF-20, and FGF-21 or fragments thereof that bind to an FGF receptor.

49. The bioreactor of claim 1, wherein the biocompatible substance is a biological matrix.

50. The bioreactor of claim 49, wherein the biological matrix comprises a polymer.

51. The bioreactor of claim 49, wherein the biological matrix is selected from the group consisting of collagen, purified proteins, purified peptides, polysaccharides, glycosaminoglycans, and extracellular matrix compositions.

52. The bioreactor of claim 49, wherein the biological matrix comprises fibrin.

53. The bioreactor of claim 49, wherein the biological matrix comprises collagen.

54. The bioreactor of claim 53, wherein the collagen is type I collagen.

55. The bioreactor of claim 53, wherein the collagen is type II collagen.

56. The bioreactor of claim 51, wherein the polysaccharides are selected from the group consisting of chitosan, alginate, dextran, hyaluronic acid, and cellulose.

57. The bioreactor of claim 1, wherein the biocompatible matrix is a synthetic matrix.

58. The bioreactor of claim 57, wherein the synthetic matrix comprises a polymer.

59. The bioreactor of claim 58, wherein the polymer is selected from the group consisting of polyesters, polyethers, polyanhydrides, polyalkylcyanoacrylates, polyacrylamides, polyorthoesters, polyphosphazenes, polyvinylacetates, block copolymers, polypropylene, polytetrafluoroethylene (PTFE), and polyurethanes.

60. The bioreactor of claim 58, wherein the polymer comprises lactic acid.

61. The bioreactor of claim 58, wherein the polymer comprises glycolic acid.

62. The bioreactor of claim 58, wherein the polymer is a copolymer.

63. The bioreactor of claim 62, wherein the copolymer comprises lactic acid and glycolic acid (PLGA).

64. The bioreactor of claim 1, wherein the biocompatible substance is biodegradable.

65. The bioreactor of claim 1, wherein the biocompatible substance is non-biodegradable.

66. The bioreactor of claim 65, wherein the non-biodegradable substance comprises a polymer selected from the group consisting of poly(dimethysiloxane) and poly(ethylene-vinyl acetate).

67. The bioreactor of claim 1, wherein the biocompatible substance is selected from the group consisting of collagen, metal, hydroxyapatite, bioglass, aluminate, bioceramic materials, hyaluronic acid polymers, alginate, acrylic ester polymer, lactic acid polymer, glycolic acid polymer, lactic acid/glycolic acid polymer, purified proteins, purified peptides, and extracellular matrix compositions.

68. The bioreactor of claim 67, wherein the biocompatible substance is a lactic acid/glycolic acid polymer.

69. The bioreactor of claim 1, wherein the biocompatible substance is associated with an implantable device.

70. The bioreactor of claim 69, wherein the device is selected from the group consisting of: a stent, a catheter, a fiber, a hollow fiber, a patch, and a suture.

71. The bioreactor of claim 69, wherein the device contains expanded polytetrafluoroethylene (ePTFE) or Dacron.

72. A method adapted for systemic delivery of a protein from a tissue site in an animal, comprising contacting the tissue site with the in situ bioreactor of any one of claims 1-18.

73. The method of claim 72, wherein the tissue site is the site of an iatrogenic injury.

74. The method of claim 72, wherein the tissue site is subcutaneous, intramuscular, intraperitoneal, or retroperitoneal.

75. The method of claim 72, wherein the tissue site is an organ.

76. The method of claim 72, wherein the animal is a mammal.

77. The method of claim 76, wherein the mammal is a human.

78. The method of claim 72, wherein subsequent to contacting the tissue site with the bioreactor, the bioreactor is supplemented with additional quantities of the first nucleic acid molecule encoding a cell growth stimulating agent.

79. The method of claim 72, wherein subsequent to contacting the tissue site with the bioreactor, the bioreactor is supplemented with additional quantities of the second nucleic acid molecule encoding a bioactive agent.

80. The method of claim 72, wherein subsequent to contacting the tissue site with the bioreactor, the bioreactor is supplemented with additional quantities of the first and second nucleic acid molecules.

81. The method of claim 72, wherein prior to contacting the tissue site with the bioreactor, the bioreactor comprises cells transduced with the first nucleic acid, the second nucleic acid, or both the first and second nucleic acids.

82. A method adapted for systemic delivery of a protein from a tissue site, comprising introducing into a tissue site of an animal an in situ bioreactor, the bioreactor comprising a first nucleic acid molecule encoding a cell growth stimulating agent, wherein the bioreactor comprises a biocompatible substance capable of infiltration by cells, and wherein a second nucleic acid molecule encoding a serum soluble protein is introduced into the bioreactor following cellular infiltration.

83. A method adapted for systemic delivery of a protein from a tissue site, comprising contacting a tissue site of an animal with an in situ bioreactor, wherein the bioreactor comprises a biocompatible substance and a first nucleic acid molecule, wherein the first nucleic acid molecule encodes a cell growth stimulating agent, and wherein said encoded cell growth stimulating agent conditions matrix infiltrating cells for uptake of a second nucleic acid molecule encoding a serum soluble protein.

84. The method of claim 83, wherein the cells are selected from the group consisting of: stem cells, macrophages, fibroblasts, and vascular cells.

85. The method of any one of claims 82-83, wherein the nucleic acid molecules are absorbed in the biocompatible substance.

86. The method of any one of claims 82-83, wherein the nucleic acid molecules are adsorbed to the biocompatible substance.

87. The method of any one of claims 82-83, wherein the nucleic acid molecules are impregnated within the biocompatible substance.

88. The method of any one of claims 82-83, wherein the biocompatible substance is a mixture of synthetic and biological materials.

89. A method adapted for systemic delivery of a protein from a tissue site, comprising introducing into a tissue site of an animal an in situ bioreactor, wherein the bioreactor comprises a biocompatible substance, a cell growth stimulating agent, and a first nucleic acid molecule encoding a serum soluble protein wherein the bioreactor is capable of infiltration by cells.

90. The method of claim 89, further comprising introducing a second nucleic acid molecule into the bioreactor.

91. The method of claim 90, wherein the second nucleic acid molecule encodes a growth factor.

92. The method of claim 90, wherein the second nucleic acid molecule is introduced subsequent to introduction of the bioreactor into the tissue site.

93. The method of claim 89, wherein the growth stimulating agent is a nucleic acid molecule encoding a growth factor.

94. The method of claim 89, wherein the growth stimulating agent is a protein.

95. The method of claim 94, wherein the protein is a growth factor.

96. The method of claim 89, wherein the protein is an angiogenic factor.

97. The method of claim 89, further comprising the step of seeding the bioreactor with cells prior to introduction to the tissue site.

98. A Bi-gene device comprising a biocompatible substance capable of cellular infiltration, a first nucleic acid molecule encoding a cell growth stimulating agent, and a second nucleic acid molecule encoding a bioactive agent.

99. The device of claim 98, wherein the biocompatible substance is a synthetic substance or biological substance.

100. The device of claim 98, wherein the device is non-biodegradable.

101. The device of claim 98, wherein the biocompatible substance comprises a substance selected from the group consisting of: PTFE, expanded PTFE, Dacron, metal, polylactic acid, polyglycolic acid, polylactic-polyglycolic acid (PLGA), collagen, bioceramic materials, alginate, and hyaluronic acid.

102. The device of claim 98, wherein the device may be supplemented with additional nucleic acid molecules.

103. A kit for the production of a device, comprising:
a) an appropriate container,
b) a biocompatible substance,
c) a first nucleic acid molecule encoding a cell growth stimulating agent; and
d) a second nucleic acid molecule encoding a bioactive agent.

104. A kit for the production of a coated device, comprising:
a) a device coated with a biocompatible substance,
b) a first nucleic acid molecule encoding a growth stimulating agent; and
c) a second nucleic acid molecule encoding a bioactive agent.
